(19) United States
(12) Patent Application Publication
(10) Pub. No.: US 2015/0367366 A1
(43) Pub. Date: Dec. 24, 2015

(54) AEROSOL DISPENSER WITH EDIBLE CARTRIDGE

(71) Applicant: AERODESIGNS, INC., Cambridge, MA (US)

(72) Inventors: David A. Edwards, Boston, MA (US); Cecily Lalor, Arlington, MA (US); Deen Bhatta, Somerville, MA (US); Anmol Giri, Somerville, MA (US); Scott Winslow, Hoboken, NJ (US); Jeffrey L. Vaughn, Newton, MA (US)

(21) Appl. No.: 14/650,211

(22) PCT Filed: Dec. 4, 2013
(86) PCT No.: PCT/US2013/073049

§ 371 Date: Jun. 5, 2015

Related U.S. Application Data
(60) Provisional application No. 61/734,257, filed on Dec. 6, 2012.
(60) Provisional application No. 61/790,472, filed on Mar. 15, 2013.
(60) Provisional application No. 61/788,212, filed on Mar. 15, 2013.

(51) Int. Cl. B05B 11/00; A61M 15/00

(52) U.S. Cl. CPC B05B 11/0054; A61M 15/0021; A61M 15/009; A61M 15/0035; A61M 2202/064

(57) ABSTRACT

An aerosolizing delivery device can include a carriage configured to be detachably connected to a mouthpiece and to receive and support an aerosolizable powder-filled cartridges in a desired position relative to the mouthpiece. The carriage can include: a housing defining a reservoir and a carriage outlet, the carriage outlet configured to permit fluid communication between the reservoir and an exterior of the carriage. A surface of the housing can be configured to define a bypass port between the housing and a surface of the mouthpiece when the carriage is assembled with the mouthpiece to form the delivery device.

### RELATED APPLICATION'S

This application claims the benefit of U.S. Patent Application No. 61/734,257, filed on Dec. 6, 2012, the benefit of 61/788,212, filed on Mar. 15, 2013, and the benefit of U.S. Patent Application No. 61/790,472, filed on Mar. 15, 2013; the entire teachings of these applications are incorporated herein by reference.

### FIELD OF THE INVENTION

The invention relates generally to aerosolized particles and devices for the containment, aerosolization, and/or delivery thereof.

### BACKGROUND

Previous researchers have demonstrated that aerosol particles can be used to deliver substances to various parts of the body. Certain designs have been proposed for utilizing these particles for drug delivery, and devices and methods have been developed for the delivery of aerosolizable products and uses thereof.

### SUMMARY

Light, consumable particles can be drawn into a user's mouth for deposition on surfaces of the mouth for consumption through transdermal surfaces and/or through the digestive tract (e.g., ingestion via intake into the stomach and gastrointestinal tract by means of enteral administration). However, when consuming particles that are sufficiently light to be drawn into a user's mouth by inhalation or exhalation, one must address the risk of those particles reaching the back of the mouth or lungs and causing coughing or other adverse events, especially when the goal is, for example, to provide taste, nourishment, dietary supplementation, and/or medicinal delivery, involving the mouth, tongue, etc.

Therefore, approaches to deliver materials to the mouth via the airborne route have largely (if not exclusively) focused on directed, non-breath-actuated delivery, where the force of the air current and size of the particles are such that particle trajectories are prima limited to with in the mouth.

We have developed an approach by which a casual or forced breathing maneuver (such as normal inhalation or exhalation) can lead to the delivery of food, drink, medicinal and/or various other particles to the mouth, in which the transport of these particles with the flowing air, to the back of the throat and to the lungs, is limited. By controlling the inertia and gravity of the particles (e.g., food particles), and by directing deposition forces, we can focus delivery of the particles towards surfaces of the mouth, not reaching the back of the throat and lungs.

In some aspects, a carriage device is provided for retaining a cartridge. The device includes a first end defining an outlet port and configured to be detachably connected to a mouthpiece; a second end defining an inlet port that is configured to align with an inlet port of the cartridge, the second end being opposed to the first end; a sidewall connecting the first end and the second end, the sidewall defining an access port that is configured to permit passage of the cartridge therethrough; a reservoir defined between the first end, second end and the sidewall, the reservoir configured to communicate with the inlet port, the outlet port and the access port and to receive the cartridge; and an access port closure connected to the sidewall and movable between an open configuration and a closed configuration. When a cartridge is disposed in the reservoir, an air flow bypass passage is formed between the cartridge and an interior surface of the device.

The device may include one or more of the following features: The access port closure is configured to rotate about a rotational axis that is parallel to a longitudinal axis of the device when moving between the open configuration and the closed configuration. The second end further comprises an opening that is configured to facilitate cartridge removal from the reservoir. The device includes the mouthpiece connected to the first end, and the cartridge disposed in the reservoir, wherein the mouthpiece, the device and the cartridge cooperate to provide a flow rate through an interior of the device of between about ten (10) liters per minute and about sixty (60) liters per minute at a vacuum pressure of about four kiloPascals. The device is configured to be detachably connected to the mouthpiece by a press fit, a magnetic retaining mechanism, a twist mechanism, a snap mechanism, a screw mechanism, a bayonet mount mechanism, or combinations thereof. The device includes the cartridge, the cartridge being at least one of edible and biodegradable and comprising a housing defining a cartridge reservoir, and an edible, aerosolizable powder disposed in the reservoir. The cartridge reservoir has a volume capacity of between about ten (10) milligrams to about two (2) grams of a product. The cartridge is formed from at least one of the group consisting of a starch, a grain-based food, a vegetable, a meat, a fruit, a dairy product, a sugary food, a nut, a confection, a plant product, processed edible products thereof, and synthetic edible products thereof. The cartridge is formed from at least one of the group consisting of chocolate, bread, fruit, sugar, meat, pasta, and processed forms thereof. The cartridge and the aerosolizable powder contained in the cartridge are comprised of at least one of the group selected from a vitamin, a mineral, and a supplement. The amount of vitamin. mineral or supplement provided by the cartridge, and the amount of vitamin. mineral, or supplement comprising the aerosolizable powder, in sum provide a quantity corresponding to an adult minimum daily requirement for the vitamin, mineral or supplement. The cartridge is biodegradable and is formed from at least one of the group consisting of a polyester, a polyhydroxyalkanoate, a polyanhydride, a polycaprolactone, a polydiaxonone, polyglycolide, a polyhydroxybutyrate, a polylactic acid, a polypropylene carbonate, a polylactic-co-glycolic acid, a poly(3-hydroxybutyrate-co-3-hydroxyvalerate), a polyvinyl alcohol, a starch derivative, a cellulose derivative, a cellulose ester, a cellophane, an enhanced biodegradable plastic, and compositional variants thereof. The channel is provided on an exterior surface of the cartridge housing, and the channel is substantially parallel to a longitudinal axis of the device and configured to be in fluid communication with the device outlet port and device inlet port when the cartridge is disposed in the device reservoir, the channel operable as an air bypass passage when the access port closure is substantially in the closed configuration.

In some aspects a carriage device for retaining a cartridge includes a first end configured to be detachably connected to a mouthpiece; an open second end opposed to the first end; an axis extending between the first end and the second end; a sidewall extending between the first end and the second end; a reservoir defined between the first end, the second end and the sidewall, the reservoir configured to receive the cartridge; a first plunger, connected to the second end, the first plunger configured to be moveable relative to the sidewall along the axis; and a second plunger, connected to the first plunger, the second plunger configured to move relative to the first plunger along the axis.

The device may include one or more of the following features: The first plunger is configured to secure the cartridge within the reservoir. The second plunger is configured to puncture the cartridge. An awl is located within the reservoir, the awl extending in parallel to the axis, wherein the awl is operable to puncture the cartridge. The sidewall includes an access port. The first plunger is releasably connected to the second end by a press fit, a magnetic retaining mechanism, a twist mechanism, a snap mechanism, a screw mechanism, a bayonet mount mechanism, or combinations thereof. The first plunger comprises a hollow cylinder defining a first cylinder end releasably connected to the second end and including an inwardly protruding lip circumscribing the inner surface substantially parallel to the first cylinder end. The second plunger comprises a second plunger first end disposed perpendicularly to the axis, and arms protruding from the second plunger first end, and wherein the arms are configured to engage the lip of the first plunger and permit movement of the second plunger along the axis. An outer surface of the first end is configured to define a bypass port between the first end and a surface of a mouthpiece when the carriage is assembled with the mouthpiece. The mouthpiece is connected to the device first end, and the cartridge is disposed in the reservoir, wherein the mouthpiece, the device and the cartridge cooperate to provide a flow rate through the device of between about ten (10) liters per minute and about sixty (60) liters per minute at a vacuum pressure of about four kiloPascals. The mouthpiece and the device are configured to be releasably connected by a press fit, a magnetic retaining mechanism, a twist mechanism, a snap mechanism, a screw mechanism, a bayonet mount mechanism, or combinations thereof. The device includes the cartridge, wherein the cartridge is at least one of edible and biodegradable and comprises a housing defining a cartridge reservoir, and an edible, aerosolizable powder disposed in the cartridge reservoir. The cartridge reservoir has a volume capacity of between about ten (10) milligrams to about two (2) grams of a product. The cartridge is formed from at least one of the group consisting of a starch, a grain-based food, a vegetable, a meat, a fruit, a dairy product, a sugary food, a nut, a confection, a plant product, processed edible products thereof, and synthetic edible products thereof. The cartridge is formed from at least one of the group consisting of chocolate, bread, fruit, sugar, meat, pasta, and processed forms thereof. The cartridge and the aerosolizable powder contained in the cartridge are comprised of at least one of the group consisting of a vitamin, mineral and supplement. The amount of vitamin, mineral or supplement provided by the cartridge, and the amount of vitamin, mineral, or supplement comprising the aerosolizable powder, provide a quantity corresponding to an adult minimum daily requirement for the vitamin, mineral, or supplement. The cartridge is biodegradable and is formed from at least one of the group consisting of a polyester, a polyhydroxyalkanoate, a polyanhydride, a polycaprolactone, a polydiaxonone, a polyglycolide, a polyhydroxybutyrate, a polylactic acid, a polypropylene carbonate, a polylactic-co-glycolic acid, a poly(3-hydroxybutyrate-co-3-hydroxyvalerate), a polyvinyl alcohol, a starch derivative, a cellulose derivative, a cellulose ester, a cellophane, an enhanced biodegradable plastic, and compositional variants thereof.

In some aspects, a carriage device for retaining a cartridge includes a first end configured to be releasably connected to a mouthpiece; a second end opposed to the first end, a sidewall extending between the first end and the second end; a partition that is disposed between the first end and the second end and extends between opposed inner surfaces of the sidewall to segregate an interior space of the carriage into a first portion and a second portion; and a piercing tool disposed in the second portion.

The device may include one or more of the following features: The first end includes legs equidistantly spaced about a circumference of the sidewall, the legs configured for engagement with a surface of the mouthpiece. The first portion is defined between the legs and the partition, and forms a reservoir configured to receive a cartridge. The partition includes openings that provide fluid communication between the first portion and the second portion. The device includes the mouthpiece releasably connected to the first end, and the cartridge disposed in the first portion, wherein the mouthpiece, the device and the cartridge cooperate to provide a flow rate through the device of between about ten (10) liters per minute and about sixty (60) liters per minute at a vacuum pressure of about four kiloPascals. The mouthpiece and the device are configured to be releasably connected by a press fit, a magnetic retaining mechanism, a twist mechanism, a snap mechanism, a screw mechanism, a bayonet mount mechanism, or combinations thereof. The device includes the cartridge, the cartridge being at least one of edible and biodegradable, the cartridge comprising a housing defining a cartridge reservoir, and an edible, aerosolizable powder disposed in the cartridge reservoir. The cartridge reservoir has a volume capacity of between about ten (10) milligrams to about two (2) grams of a product. The cartridge is formed from at least one of the group consisting of a starch, a grain-based food, a vegetable, a meat, a fruit, a dairy product, a sugary food, a nut, a confection, a plant product, processed edible products thereof, and synthetic edible products thereof. The cartridge is formed from at least one of the group consisting of chocolate, bread, fruit, sugar, meat, pasta, and processed forms thereof. The cartridge and the aerosolizable powder contained in the cartridge are comprised of at least one of the group consisting of the group consisting of a vitamin, a mineral, and a supplement. The amount of vitamin, mineral, or supplement provided by the cartridge, and the amount of vitamin, mineral, or supplement comprising the aerosolizable powder provide a quantity corresponding to an adult minimum daily requirement for the vitamin, mineral or supplement. The cartridge is biodegradable and is formed from at least one of the group consisting of a polyester, a polyhydroxyalkanoate, a polyanhydride, a polycaprolactone, a polydiaxonone, a polyglycolide, a polyhydroxybutyrate, a polylactic acid, a polypropylene carbonate, a polylactic-co-glycolic acid, a poly(3-hydroxybutyrate-co-3-hydroxyvalerate), a polyvinyl alcohol, a starch derivative, a cellulose derivative, a cellulose ester, a cellophane, an enhanced biodegradable plastic, and compositional variants thereof.

In certain embodiments of the aerosolizing delivery device, an aerosolizing delivery device configured to be connected to a carriage, includes: a first member defining a first interior volume, an inlet, an outlet, an aerosol flow passage, and a portion configured to be connected to the carriage; and a deflection member configured to be received in the mouth of a user, spaced apart from a plane that includes the first member outlet, positioned to redirect aerosol flow exiting the outlet toward one or more sides of the user's mouth.

In certain embodiments, the carriage includes a reservoir, and a cartridge is disposed in the reservoir.

In certain embodiments of the aerosolizing delivery device, the cartridge defines a second interior volume and detachably connectable to and in fluid communication with the first member, the cartridge defining at least one cartridge air inlet and at least one cartridge outlet, wherein the cartridge air inlet is in fluid communication with the second interior volume, and the outlet is in fluid communication with the second interior volume and the first member aerosol flow passage.

In certain embodiments of the aerosolizing delivery device, the cartridge is indirectly detachably connected to the first member via the carriage, or directly detachably connected to the first member by any one of a press fit, a twist mechanism, a snap mechanism, screw mechanism, bayonet mechanism, or combinations thereof.

In certain embodiments of the aerosolizing delivery device, the cartridge further includes at least one cartridge bypass port.

In certain embodiments of the aerosolizing delivery device, the cartridge has a volume capacity of about ten (10) milligrams to about two (2) grams of an aersolizable product.

In certain embodiments of the aerosolizing delivery device, the cartridge contains an aerosolizable product.

In certain embodiments of the aerosolizing delivery device, the aerosolizable product is at least one of a food product, an energy supplement, a vitamin and/or mineral supplement, a pharmaceutical compound, an over-the-counter pharmaceutical compound, a nutraceutical, a sleep-aid compound, a weight-loss compound, or an oral health compound.

In certain embodiments of the aerosolizing delivery device, the cartridge contains an aerosolizable product.

In certain embodiments of the aerosolizing delivery device, the cartridge contains at least one of a food product, an energy supplement, a pharmaceutical compound, an over-the-counter pharmaceutical compound, a nutraceutical, a sleep-aid compound, a weight-loss compound, or an oral health compound.

In certain embodiments of the aerosolizing delivery device, the device is at least one of edible or biodegradable.

In certain embodiments of the aerosolizing delivery device, at least one of the first member and the deflection member is at least one of edible or biodegradable.

In certain embodiments of the aerosolizing delivery device, the cartridge is at least one of edible or biodegradable.

In certain embodiments of the aerosolizing delivery device, the cartridge is edible and is formed of at least one of a starch, a grain-based food, a vegetable, a meat, a fruit, a dairy product, a sugary food, a nut, a confection, a plant product, an energy supplement, a vitamin and/or mineral supplement, a pharmaceutical compound, an over-the-counter pharmaceutical compound, a nutraceutical, a sleep-aid compound, a weight-loss compound, an oral health compound, processed edible products thereof, synthetic edible products thereof, or combinations of edible products thereof.

In certain embodiments of the aerosolizing delivery device, the cartridge is edible and is formed of at least one of chocolate, bread, fruit, sugar, meat, bread, pasta, processed forms thereof, or combinations thereof.

In certain embodiments of the aerosolizing delivery device, the cartridge is biodegradable and is formed of at least one of a polyester, a polyhydroxyalkanoate, a polyanhydride, a polycaprolactone, a polydiaxonone, a polyglycolide, a polyhydroxybutyrate, a polylactic acid, a polypropylene carbonate, a polylactic-co-glycolic acid, a poly(3-hydroxybutyrate-co-3-hydroxyvalerate), a polyvinyl alcohol, a starch derivative, cellulose esters, a cellophane, an enhanced biodegradable plastic, compositional variants thereof, or combinations thereof.

### BRIEF DESCRIPTION OF THE DRAWINGS

The advantages of the disclosure described below, as well as further advantages of the disclosure, can be better understood by reference to the description taken in conjunction with the accompanying figures, in which:

FIG. 1 is a side view of a particle delivery device including a carriage connected to a mouthpiece and illustrating a cartridge to be inserted into the carriage.

FIG. 2 is a rear view of the particle delivery device of FIG. 1 illustrating the position of the cartridge when loaded into the carriage.

FIG. 3 is a perspective view of the particle delivery device of FIG. 1 illustrating a carriage door in a closed position.

FIG. 4 is a cross-sectional perspective view of the particle delivery device of FIG. 1 as seen along line 2-2 of FIG. 3.

FIG. 5 is an exploded perspective view of the carriage depicted in FIG. 1

FIG. 6 is a bottom perspective view of the carriage depicted in FIG. 1 with the carriage door in an open position.

FIG. 7 is side perspective view of the particle delivery device of FIG. 1 with the carriage door in an open position.

FIG. 8 is side perspective view of the particle delivery device of FIG. 1 with the carriage door in an open position and a cartridge disposed in the reservoir of the carriage.

FIG. 9 is a bottom perspective view of the particle delivery device of FIG. 1 with the door in a closed position and a cartridge disposed in the reservoir of the carriage.

FIG. 10 is a cross-sectional perspective view of the cartridge of FIG. 11.

FIG. 11 is a top perspective view of the cartridge depicted in FIG. 1.

FIG. 12 is a bottom perspective view of the cartridge depicted in FIG. 1 FIG. 13 is a front side view of another embodiment of a carriage.

FIG. 14 is a side view of the carriage of FIG. 13.

FIG. 15 is a top perspective view of the carriage of FIG. 13.

FIG. 16 is an exploded perspective view of the carriage of FIG. 13.

FIG. 17 is a cross-sectional perspective view of the carriage of FIG. 13 with the carriage reservoir empty.

FIG. 18 is a cross-sectional perspective view of the carriage of FIG. 13 with a cartridge disposed in the carriage reservoir.

FIG. 19 is a cross-sectional side view of the carriage of FIG. 13 illustrating the carriage in a loading configuration.

FIG. 20 is a cross-sectional side view of the carriage of FIG. 13 illustrating the carriage in a piercing configuration.

FIG. 21 is a cross-sectional side view of the carriage of FIG. 13 illustrating the carriage in a use configuration.

FIG. 22 is a front side view of another embodiment of a particle delivery device including a cartridge supported by another embodiment carriage so that the carriage is adjacent the mouthpiece.

FIG. 23 is a top perspective view of the carriage depicted in FIG. 22.

FIG. 24 is a bottom perspective view of the carriage depicted in FIG. 22.

FIG. 25 is a bottom perspective view of another embodiment of a particle delivery device carriage.

FIG. 26 is a top perspective view of the carriage of FIG. 25.

### DETAILED DESCRIPTION

Aerosolized particles small enough to become airborne but too large to be inhaled into the bronchial pathways and lungs retain a sufficient surface area to volume ratio to be solubilized effectively in the mouth. Therefore, a natural breathing process to aerosolize particles can be a particularly effective way to enhance mouth absorption of certain active ingredients. Described herein are various embodiments of an aerosol delivery device designed to use a natural breathing process to aerosolize a consumable product and deliver to the mouth for oral absorption and/or digestive solubilization. Such devices are designed to limit product delivery to the user's throat or lungs, allow for multiple use applications with the same oral delivery device, and provide for enhanced user experience (choice of consumable product to be used with a single device and environmentally low-impact and/or edible devices).

A variety of forces can be used to generate an aerosol from a fluid or dry powder to cause it to move through an aerosol delivery device. These include a user's inhalation/exhalation, aspiration/expiration, shaking or vibration forces, and/or external power sources (e.g., compressed air, electric fans, motors, etc.). Particle size is important to the delivery system. Particles should be small enough to remain airborne during casual breathing, but large enough to be directed and deposited primarily in the mouth while limiting coughing, throat and lung deposition, or other adverse situations. Secondly, it is beneficial that pathways of aerosol particles through the device and out of the mouthpiece are directed away from the back of the throat.

Molecules of consumable, aerosolized products generally absorb in the mouth and the digestive tract via a three-step process. The first step is dissolution or release from a dosage form, the second is diffusion or convection from the site of dissolution to the absorptive mucosa, and the third is active or passive transport across the mucosa into the bloodstream. As used herein, mucosa (or mucosae in plural form) includes mucous membranes that are linings of mostly endodermal origin, covered in epithelium, which are involved in absorption and secretion. Mucosae border cavities, openings and lumen that are exposed to the external environment and internal organs, and can be contiguous with skin in a person's mouth. For those molecules that may absorb across a mucosal barrier at a kinetically effective rate (as determined by hydrophobicity, charge, and molecular size—the more hydrophobic, neutral, and small, the better), the actual rate at the anatomical site of absorption into the bloodstream, once the dosage form is placed in the mouth, is controlled to some degree by the first two steps in the above process, and to some degree by the speed of the digestive process itself.

Consumables (e.g., foods, supplements, or drugs) can be deliverable as chewable solids, liquids, pill, gum, soluble media (e.g., strips) and as a fine powder form that gets distributed on the surfaces of the mouth. Common delivered dosage forms begin to dissolve in the mouth and are swallowed. The active ingredient then diffuses through the mucosa. Depending on the amount of molecule contact with the mucosa, this diffusion may take a significant amount of time in which a user may swallow significant dosage portions of an active ingredient before it has the chance to absorb through the mouth. In cases where the diffusion distance to the tongue is small, and the likelihood of swallowing is small (e.g., with dissolvable strips), mouth-versus-gut absorption is still influenced by the time of dissolution. Increased times for dissolution in your mouth can correlate with more occasions to swallow during the dissolution, and the increased likelihood that dissolved ingredients are swept into the gut and subjected to digestive processes.

Our approach is based, at least in part, on the realization of a new form of aerosolizable product, devices for the delivery of aerosolizable products, and methods and uses thereof. More specifically, the delivery technology and approach is directed to aerosolized particles (i.e., food products) and a particle delivery method and device designed to generate and deliver such products to a subject. Such devices can deliver food substances or other particles into the mouth by aerosol wherein the aerosol cloud is generated through a natural inspiration or expiration maneuver and wherein the design of the mouthpiece of the device is such that the airborne particles (e.g., food particles) are diverted away from the back of the throat to limit entry into the respiratory system. Although described with respect to delivery of food products, the devices and methods discussed herein can be used for generation and delivery of other products (e.g., medicinal products, flavorings, nutritional supplements, etc.).

In some embodiments, the combination of appropriate particle size and device-directed aerosol flow path allows for consumable particles (e.g., food particles) being deposited primarily in the mouth (and onto the tongue, palate, etc.) rather than at the back of the throat or into the respiratory tract. In some embodiments, aerosol flow paths are directed to the sides of a user's mouth and not into the lungs, substantially eliminating deposition in regions of the respiratory tract on or near the throat and bronchial airways.

Various embodiments are contemplated and consider certain parameters for effective device function. Physical design differences of the device affect fluid flow properties, such as typical fluid resistances or pressure drops across sections of the device (e.g., a pressure drop over the consumable product, which gives rise to its aerosolization). For particulate or liquid consumables, rates of acceleration, particle velocities, or time of flight durations in sections of the device or upon emission, (e.g., the time for an aerosol to displace from an inlet to an outlet or the velocity it has upon emission) are considered. Also contemplated are aerosol properties, including size, shape, orientation, particle concentration, particle-size distribution, homogeneity, individual particle velocities, and overall (e.g., center-of-mass) aerosol velocities (e.g., the number of consumable-aerosol particles of a given size range, per unit volume of air, upon emission of the aerosol toward a consumer), and/or typical aerosol emission parameters, including the overall flow speed(s) and direction(s) of emitted aerosol and the locations and rates of deposition, relative to the device or consumer (e.g., specific mouth surfaces toward which the aerosol is emitted, on which the aerosol particles are most likely to deposit first).

For any generalized aerosol generation/delivery device, various design parameters and fluid flow properties determine the proportions of the consumable, aerosolizable product emitted from the device. For example, aerosol flow paths that are longer (e.g., between an inlet and an outlet), thinner (e.g., have a smaller cross-sectional area), more tortuous (e.g., have a more sinuous path); and/or more encumbered (e.g., more/larger elements like internal partitions, in closer proximity to the flow path) generally increase the time it takes for an aerosolizable product to reach a user, and generally increase the likelihood that particles (or a proportion of particles) settle before being emitted from the device. This may reduce the proportion of initial product that is ultimately delivered in aerosol form with the desired properties (or decreases the probability that the initial product is ultimately delivered in aerosol form with the desired properties).

A particle delivery device 100 is described that includes features, devices, or elements for containing or receiving aerosolizable particles, and a fluid flow passage extending between an inlet and an outlet. In some embodiments, the device is intended to deliver a consumable aerosolizable product to surfaces within a consumer's mouth. The design of the device, including the shapes, sizes, and orientations of its various components, may have significant impact on a usable consumable product, including the degree of aerosolization, product flow through sections of the device, and emission characterization from the device. In different embodiments, fluid flow passages of the device can be designed with different physical parameters, for example, different air paths, aerosol flow paths, and flow path lengths, different tortuosities (e.g., flow path complexities), different geometries (e.g., inlet or outlet cross-sectional areas and lengths), air flow bypass ports to optimize performance and flow characteristics of the device, and/or different orientations and positions of aerosol flow, bypass port and air inlets and outlets relative to each other and/or the user. The device design parameters thus determine, at least in part, the effectiveness of the system overall in delivering a desired substance to a consumer.

The design of the device can be limited by the rate of aersolization upon actuation and time to transfer to the user's mouth. If, for example, the device design requires an aerosolization time and/or aerosol transfer time outside of a predetermined threshold adequate for the device, the qualities of the aerosolized product may be suboptimal. As particle size and quality are relatively limited by natural physical characteristics of aerosolizable powders and liquids generally, design constraints are more likely to be imposed on the parameters of the device. Nevertheless, for certain relationships among the design parameters and the associated impact on device function, changes may be made to other design parameters to improve function. In general, embodiments described herein appropriately balance the design constraints in such a way as to allow for practical emission of an aerosol product for consumption.

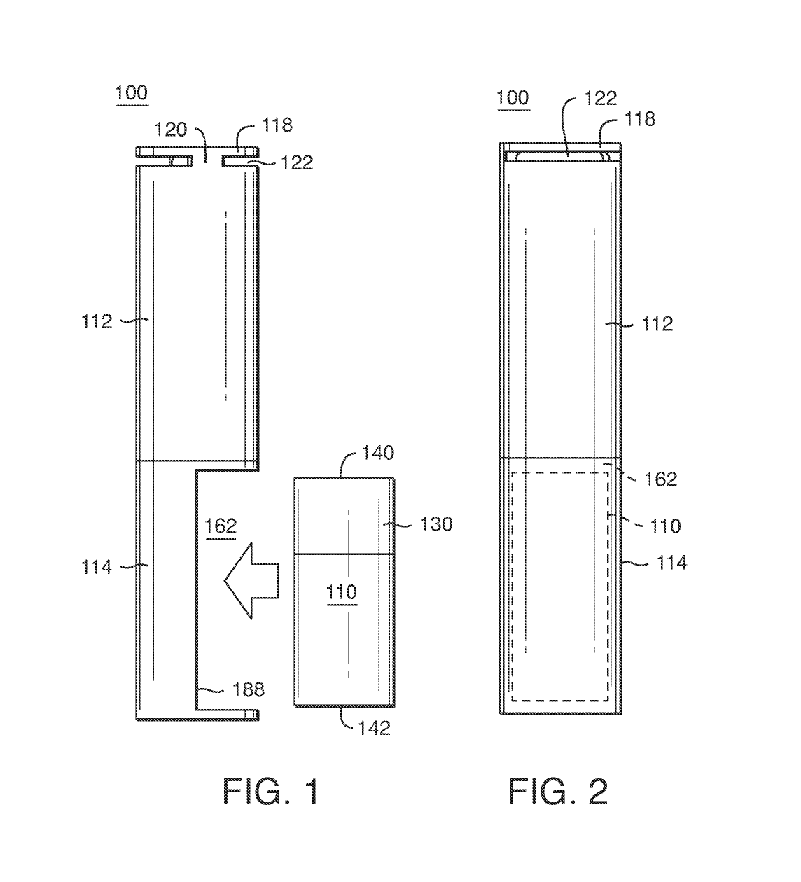

Referring to FIGS. 1-2, a particle delivery device 100 includes a mouthpiece 112 and a carriage 114 that is detachably connected to the mouthpiece 112. The carriage 114 is used to support an edible, particle-filled cartridge 110 in a position adjacent to the mouthpiece 112. The particle delivery device 100 is sized such that a user can easily hold the device in one hand while using the device 100 to generate and deliver an aerosolized product.

Referring also to FIG. 4, the mouthpiece 112 is a rigid, hollow, cylindrical member. The mouthpiece 112 defines a fluid flow passage 116 extending from an inlet 124 to the outlet 122 of the mouthpiece 112 along a longitudinal axis 126 of the mouthpiece 112. In some embodiments, the mouthpiece 112 can have a non-circular cross-section. For example, some mouthpieces have, for example, square, rectangular, or oval cross-sections.

An airflow directing or deflection member 118 is supported at an end of the mouthpiece 112 (e.g., an outlet end of the mouthpiece 112) using bridges 120. The bridges 120 position the airflow directing member 118 in a location spaced apart from a plane of an outlet 122 of the mouthpiece 112.

In some embodiments, the airflow directing member 118 is a deflection member that may take any of a variety of forms (not necessarily that of a disc), in order to divert the airflow exiting the mouthpiece 112 and entering the mouth, away from a straight trajectory toward the throat and lungs. For example, there may be one or more openings near the top of a mouthpiece 112, which may be offset relative to each other, at different heights, of different sizes, of different areas, etc., which maintain the general blockage of the direct linear path toward the back of the mouth, and generally divert the airflow and aerosol such that it goes in more lateral directions.

In some embodiments, airflow directing member 118 is a thin disc with a flat surface that is generally perpendicular to the longitudinal axis 126 of the mouthpiece 112 and in opposition to the general airflow direction (e.g., along the longitudinal axis 126) in the mouthpiece 112. In some implementations, the airflow directing member 118 changes the airflow direction of air exiting the mouthpiece 112 to a direction that is angled relative to the longitudinal axis 126. For example, in some implementations, the airflow directing member 118 redirects air exiting the mouthpiece to a direction that is substantially transverse to the longitudinal axis 126 (e.g., directing the flow toward the sides of the mouth). In some cases, the disc may be mounted to the mouthpiece via one or more “bridges” 120, which may, for example, hold the disc slightly above, below, or at the same level as the edge of the mouthpiece, allowing air, and the aerosolized product to pass around the disc. In various embodiments, the disc may have a diameter smaller, equal to, or larger than the opening of the mouthpiece. Additionally, the disc may be of any desired shape, for example, an elliptical shape or round shape. The airflow directing member 118 redirects the aerosol to the sides of the mouth (e.g. top, bottom, left, and right surfaces within the mouth), thereby limiting flow of the aerosol toward the throat where it might elicit a coughing reflex. Instead, the aerosolized product deposits on the tongue or other parts of the mouth where it can be sensed and appreciated rather than carried deeper into the respiratory tract. In some embodiments, airflow directing member 118 is of a different shape, size, and/or design but similarly serves to redirect the aerosolized product so as to limit the coughing reflex and/or to enhance the taste experience. Testing of a variety of disc sizes and positions has shown that these two parameters can impact likelihood of coughing. For example, it was found in preliminary tests that a disc whose diameter is roughly equal to that of the external diameter of the mouthpiece, and that is placed close to the mouthpiece, is generally more effective in redirecting the aerosol and limiting coughing, than one whose diameter is roughly equal to that of the internal diameter of the mouthpiece (thus smaller) and that is placed at a greater distance from the mouthpiece (leaving a larger space for the aerosol to pass through).

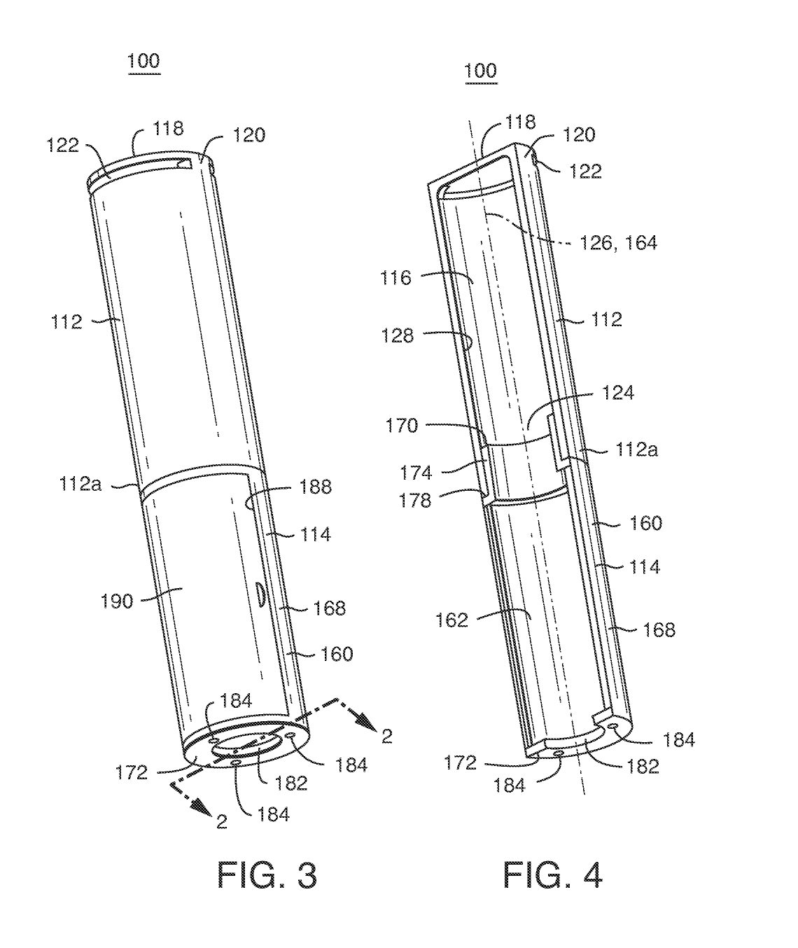

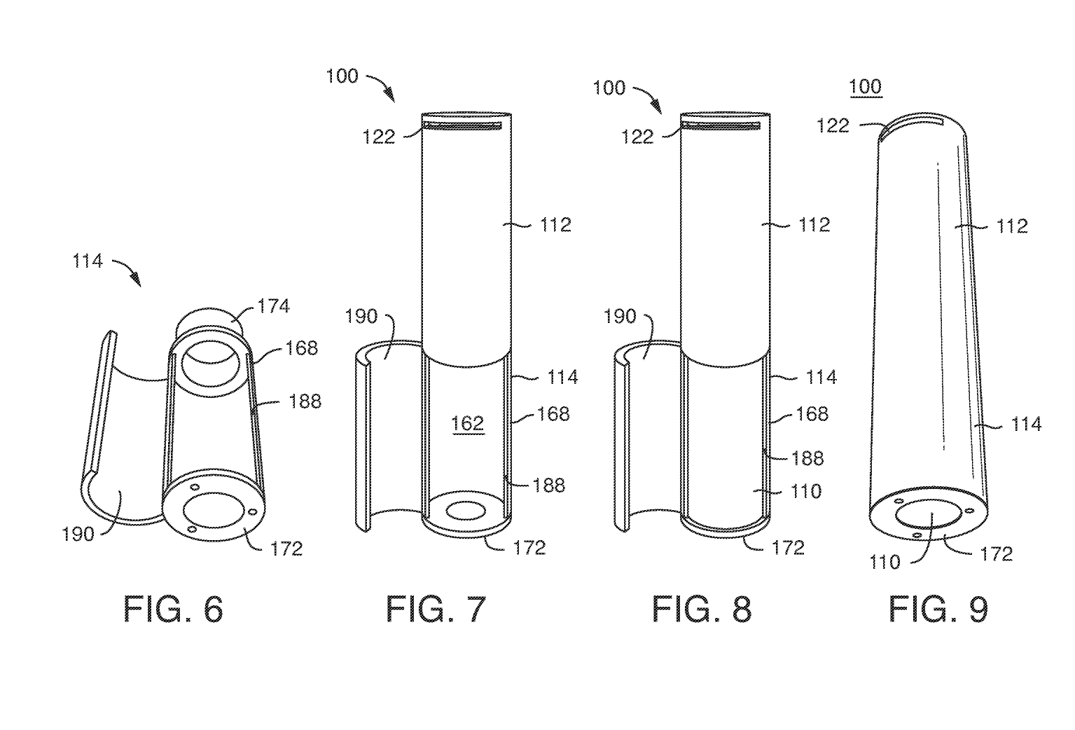

Referring to FIGS. 3-9, the carriage 114 is detachably connected to an opposed end (e.g., an inlet end 112a) of the mouthpiece 112 relative to the airflow directing member 118, and is used to retain the particle-containing cartridge 110 in a desired position relative to the mouthpiece 112. The carriage 114 can be detached from the mouthpiece and reattached, or replaced with a different carriage.

The carriage 114 is a hollow, cylindrical member. The carriage 114 includes an annular sidewall 168, an open first end 170 and a closed second end 172 opposed to the first end 170. The sidewall 168, the first end 170 and the second end 172 cooperate to define an internal reservoir 162 that receives and stores the cartridge 110. A longitudinal axis 164 of the carriage 114 extends through centers of the first and second ends 170, 172. In this embodiment, the sidewall 168 has a cross-sectional shape and outer dimension that corresponds to the cross-sectional shape and outer dimension of the mouthpiece 112. However, in some embodiments, the sidewall 168 may have a cross-sectional shape and/or outer dimension that is different from the cross-sectional shape and outer dimension of the mouthpiece 112.

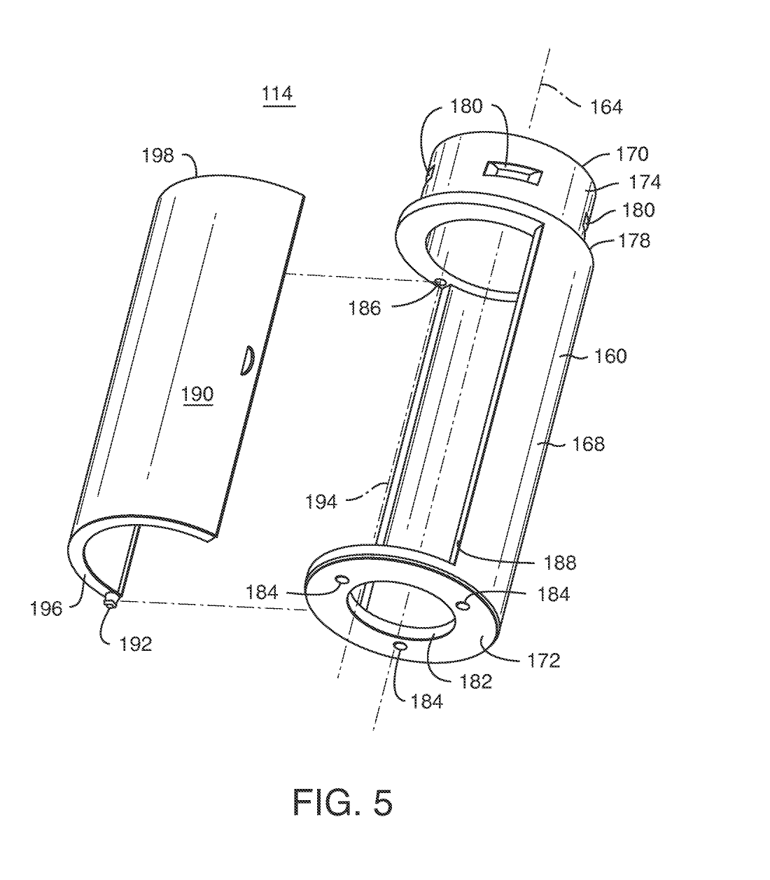

The carriage 114 includes an insertion portion 174 at the first end 170. The insertion portion 174 has a reduced outer dimension relative to the sidewall 168, whereby a shoulder 178 is provided between the insertion portion 174 and the sidewall 168. The outer dimension 174 of the insertion portion 174 corresponds to the inner dimension of the mouthpiece 112, whereby the insertion portion 174 can be inserted into the mouthpiece inlet 124. An outer surface of the insertion portion 174 is provided with circumferentially-extending grooves 180 (FIG. 5) that receive protrusions (not shown) formed on an inner surface 128 of the mouthpiece 112 adjacent to the mouthpiece inlet 124. The grooves 180 and mouthpiece protrusions cooperate to connect the carriage 114 to the mouthpiece 112. The grooves 180 and mouthpiece protrusions are arranged so that the mouthpiece inlet end 112a abuts the shoulder 178 when the carriage 114 is connected to the mouthpiece 112.

The carriage second end 172 includes a central opening 182 and peripheral openings 184 disposed between the central opening 182 and the sidewall 168. The peripheral openings 184 are equidistantly spaced from each other, and are shaped, dimensioned and arranged to correspond to openings 146 formed on the cartridge 110 (discussed below). While the peripheral openings 184 permit free flow of air to inlets formed in the cartridge, the central opening 182 permits access to the cartridge 110 when loaded in the reservoir 162, and can be used to facilitate removal of the cartridge from the reservoir 162.

Referring to FIG. 5, the sidewall 168 includes a sidewall opening 188 through which the cartridge 110 can be inserted into the reservoir 162, and a door 190 disposed within the sidewall opening 188 that is used to selectively open and close the sidewall opening 188. The sidewall opening 188 has a generally rectangular shape, and extends substantially along the full length of the sidewall 168 in an axial direction of the sidewall 168, and extends along about half the sidewall circumferential dimension in a circumferential direction of the sidewall 168. The door 190 has a shape and dimension that correspond to the shape and dimension of the sidewall opening 188. Opposed axial edges 196, 198 of the door 190 include outwardly protruding pins 192 that are received within pin openings 186 formed in an edge of the sidewall opening 188. In use, the door pivots on the pins 192 about a pin axis 194 that is parallel to the carriage longitudinal axis 164 between an first position in which the sidewall opening is open and a second position in which the sidewall opening is closed.

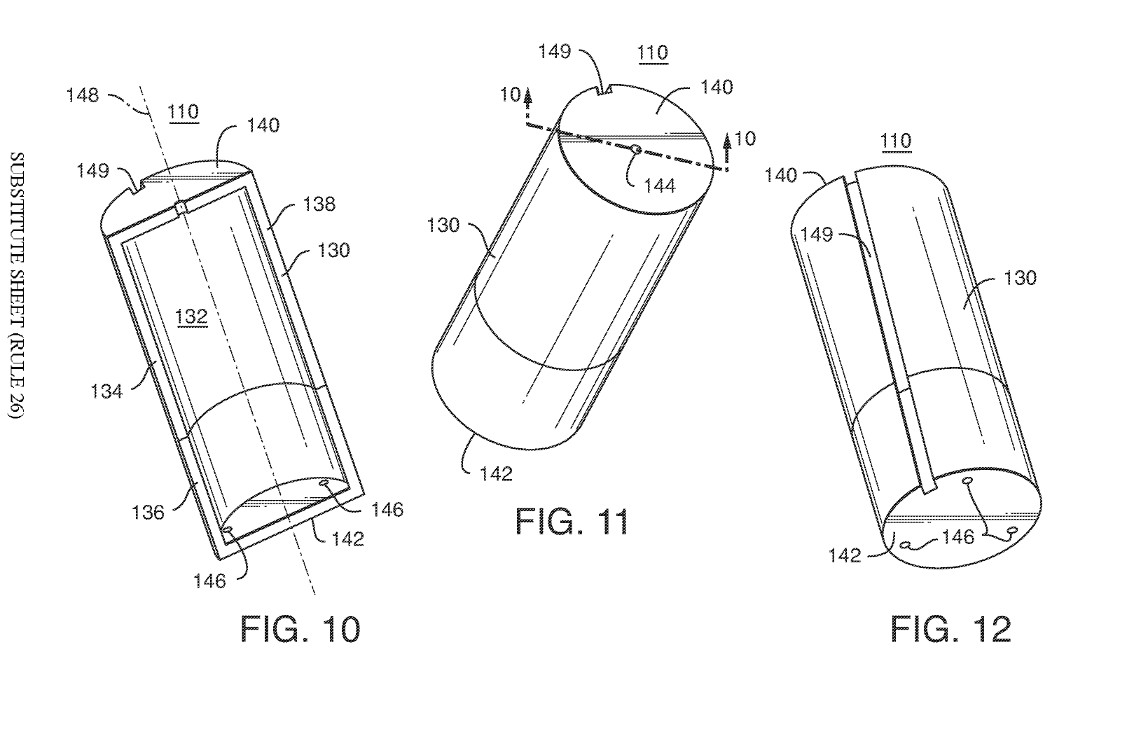

Referring to FIGS. 10-12, the cartridge 110 includes a hollow, cylindrical housing 130 that defines an internal reservoir 132 that receives and stores the aerosolizable product. The housing 130 includes an annular sidewall 138, a closed first end 140 and closed second end 142 opposed to the first end 140. A longitudinal axis 148 of the cartridge 110 extends through centers of the first and second ends 140, 142. In some embodiments, the housing 130 may be manufactured in two sections 134, 136 (which may, alternatively, be equal halves) that are fixed together to form an integral unit after the reservoir 132 has been filled. The cartridge first end 140 includes a central opening 144 that communicates with the reservoir 132 and serves as air outlet when the cartridge 110 is disposed within the carriage 114. The second end 142 includes openings 146 that communicate with the reservoir 132 and serve as air inlets. The air inlet openings 146 are located between the center and peripheral edge of the second end 142. The air inlet openings 146 are equidistantly spaced apart from each other. Although three air inlet openings 146 are illustrated, fewer or more air inlet openings 146 can be provided. In some implementations, the cartridge openings 144, 146 may be closed via product packaging (e.g., a product wrapper or peel-off tapes) to permit transport and storage without loss of the aerosolizable product.

In some embodiments, an exterior surface of the cartridge 110 includes a channel 149 that extends between the first end 140 and the second end so as to be substantially parallel to the longitudinal axis of the cartridge 148. When the cartridge 110 is disposed in the carriage reservoir 162, the channel 149 provides an air bypass passage between the outer surface of the cartridge 110 and a reservoir surface. The air bypass passage permits air to flow from the openings 182, 184 in the carriage second end 172 to the carriage open first end 170 without flowing through the cartridge reservoir 132.

Although the cartridge 110 is described herein as including cartridge openings 144, 146 which serve as air inlet ports and air outlet ports, respectively, the cartridge is not limited to this configuration. For example, in some embodiments, the cartridge is formed without the openings 144, 146 (e.g. as a completely sealed unit), and a tool (not shown) is provided that serves to punch openings in the cartridge immediately prior to insertion in the device 100.

In certain embodiments, the cartridge 110 is edible and manufactured from a starch, a grain-based food (for example, bread, pasta, etc.), a vegetable, an animal protein (for example, meat, eggs, etc), a fruit, a dairy product, a sugary food, a nut, a confection (for example, sugar, chocolate, etc.), a plant product, an energy supplement, a vitamin and/or mineral supplement, a pharmaceutical compound, an over-the-counter pharmaceutical compound, a nutraceutical, a sleep-aid compound, a weight-loss compound, or an oral health compound, processed edible products thereof, synthetic edible products thereof, and/or combinations of edible products, etc. In certain embodiments, the cartridge internal reservoir 132 is at least partially filled with the aerosolizable product (e.g., aerosol powder) discussed below.

For example, in some embodiments, the cartridge is formed of chocolate and filled with a chocolate aerosolizable powder. In other embodiments, the cartridge and the aerosolizable powder it contains are both formed of a vitamin and/or mineral supplement. This can be advantageous for vitamins and/or minerals for which humans have a large minimum daily requirement. If the volume of the reservoir 132 is insufficient to contain enough aerosolizable powder to deliver the minimum daily requirement, the deficit can be provided by including the vitamin and/or mineral in the material used to form the edible cartridge housing.

Referring to FIGS. 1-2 and 7-9, in use, the carriage 114 is connected to the mouthpiece 112 to form the particle delivery device 100. The door 190 is opened, and a cartridge 110 is inserted through the sidewall opening 188 into carriage reservoir 162 so that the cartridge air outlet 144 faces and is in fluid communication with the mouthpiece inlet 124 and so that the cartridge air inlet 146 is aligned with and in fluid communication with the central opening 182 in the carriage second end 172. After insertion of the cartridge 110, the door 190 is closed. In this configuration, the mouthpiece 112, the carriage 114 and the cartridge 110 together define a flow path through the device 100. Thus, when a user places the outlet 122 of the mouthpiece 112 in his or her mouth and inhales, air flows into the cartridge reservoir 132 through the carriage central opening 182 and the cartridge inlets 146. Air then flows from the cartridge reservoir 132 through the cartridge air outlet 144, through the carriage open first end 170 and into the inlet 124 of the mouthpiece 112. Air is drawn into the mouthpiece 112 through the mouthpiece inlet 124. The air flows along the fluid flow passage 116, and exits the mouthpiece 112 via the mouthpiece outlet 122. Contact with the airflow directing member 118 deflects the air flowing out of the mouthpiece 112, redirecting it in a direction that is angled relative to a longitudinal axis 126 of the mouthpiece 112. For example, in the illustrated embodiment, the airflow directing member 118 redirects the air flowing out of the mouthpiece 112 in a direction that is substantially perpendicular to a longitudinal axis 126 of the mouthpiece 112, but the deflection angle can be greater or lesser than 90 degrees.

A user operates a particle delivery device 100 by loading the device 100 (e.g., placing a cartridge 110 in the reservoir 162 of the carriage 114 (the carriage 114 can be connected to the mouthpiece either before or after loading the cartridge into the carriage 114), bringing the device 100 to the user's mouth, and inhaling through the mouthpiece 112 thereby causing air to enter the carriage 114 and mouthpiece 112 through the air passageways. The air aerosolizes the powder present in the cartridge reservoir 132. The aerosolized powder subsequently enters the user's mouth via the mouthpiece 112.

In some embodiments, a user places his/her tongue near an outlet (or inhaler orifice), for example outlet 122, in order to alter the speed and/or direction of the aerosol emitted from the device. In some cases, the user may position the outlet such that the aerosol is emitted toward the sublingual area. In some cases, the user may position the outlet such that the aerosol is emitted toward to lower side of the tongue, with the tongue in an elevated position (i.e. with the tip of the tongue generally closer to the top of the mouth than a region of the tongue closer to the throat). In some cases, an inspiratory or sipping maneuver under such conditions will cause aerosol particles to enter the mouth and divert to a desired surface or material within the mouth (e.g., the sides of the mouth, the top of the tongue, saliva, taste buds). In some cases, such conditions will limit undesirable side effects, such as coughing.

In some embodiments, a user places his/her teeth near an outlet (or inhaler orifice), for example outlet 122, in order to alter the speed and/or direction of the aerosol emitted from the device. In some cases, under such conditions, aerosol particles with hygienic, “freshening”, or other qualities are thus diverted toward surfaces where these particles can be most beneficial (e.g., gum surfaces).

In some embodiments, other physiological members are used to favorably alter the speed and/or direction of the aerosol emitted from the device.

In some embodiments, an aerosol is generated by an expiratory breathing maneuver, in which air emitted by a user either directly or indirectly causes a consumable product to aerosolize.

In some embodiments, the aerosol is generated at a particular point in time or over a small interval of time corresponding to a specific activation step, and/or the aerosol is generated by a user-dependent step. For example, in some cases aerosol generation is associated with one or more inhalation maneuvers by the user. In many of these embodiments, the product is in a solid state, and may be a substantially dry powder.

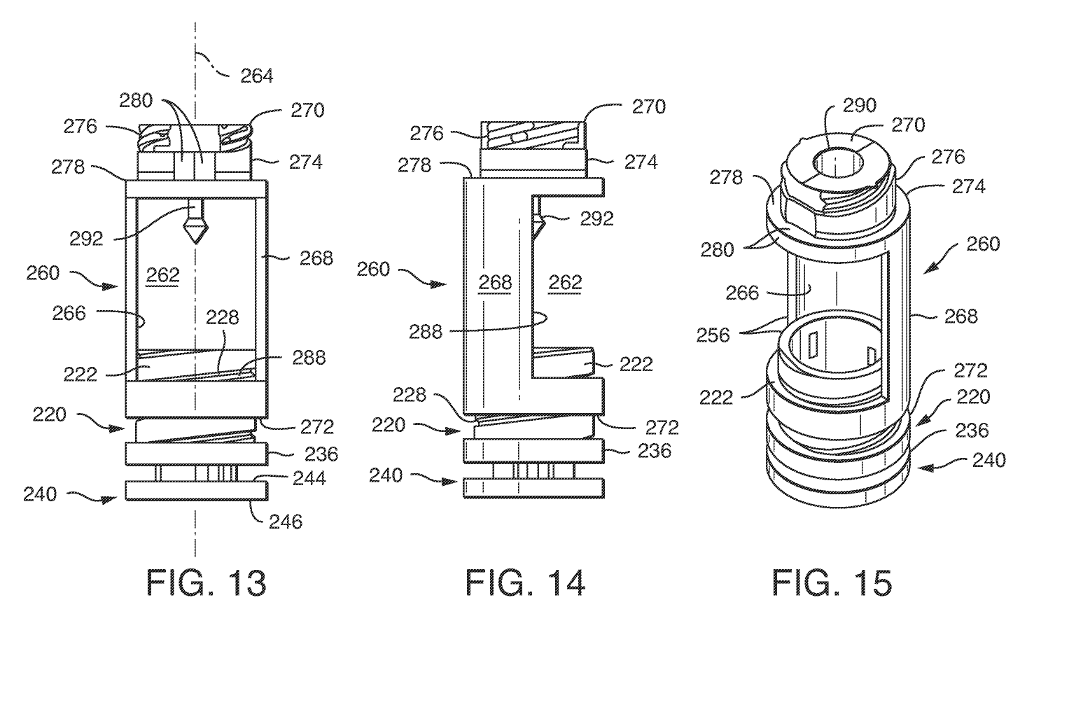

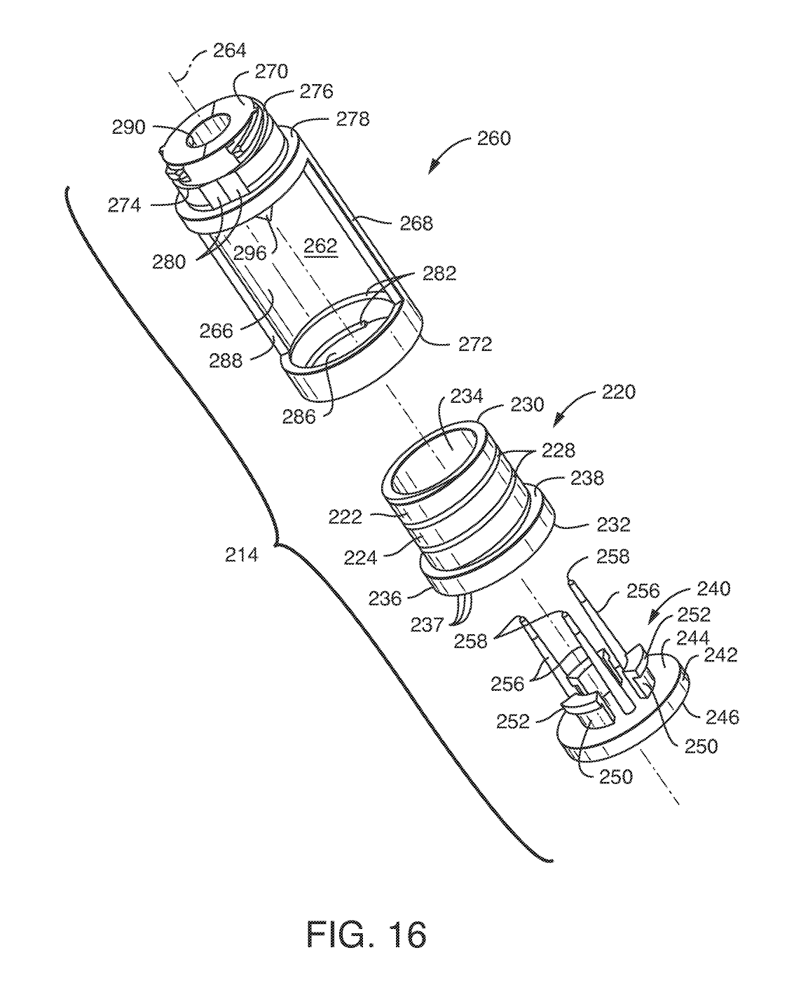

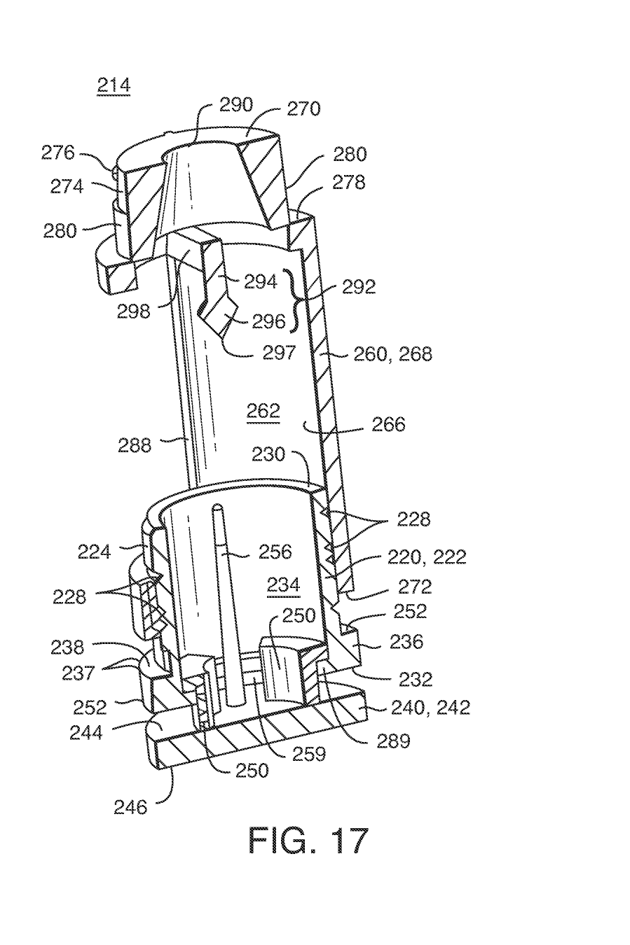

Referring to FIGS. 13-21, another carriage 214 can be used with the mouthpiece 112 and includes a hollow, cylindrical housing 260, a main plunger 220 that is received in an axial end 272 of the housing 260, and a spiked plunger 240 that is received in an axial end 232 of the main plunger 220.

Referring to FIGS. 13-16, the housing 260 includes an annular sidewall 268, an open first end 270 and an open second end 272 opposed to the first end 270. The sidewall 268, the first end 270 and the second end 272 cooperate to define an internal reservoir 262 that receives and stores the cartridge 110. A longitudinal axis 264 of the housing 260 extends through centers of the first and second ends 270, 272. The sidewall 268 has a cross-sectional shape and outer dimension that corresponds to the cross-sectional shape and outer dimension of the mouthpiece 112.

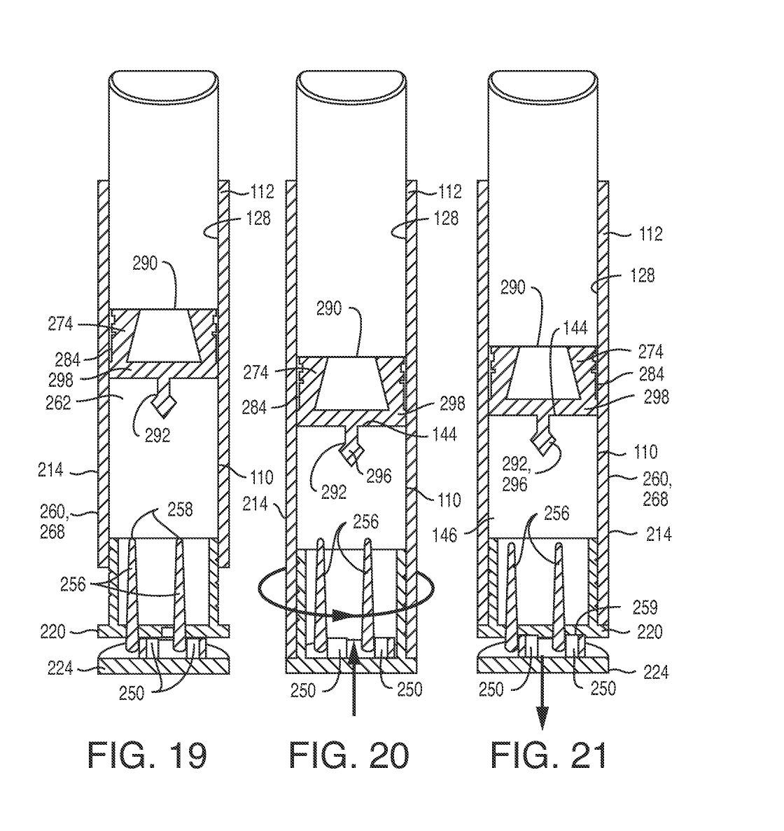

The housing 260 includes an insertion portion 274 at the first end 270. The insertion portion 274 has a reduced outer dimension relative to the sidewall 268, whereby a shoulder 278 is provided between the insertion portion 274 and the sidewall 268. The outer dimension of the insertion portion 274 corresponds to the inner dimension of the mouthpiece 112, whereby the insertion portion 274 can be inserted into the mouthpiece inlet 124. At the first end 270, an outer surface of the insertion portion 274 is provided with helical threads 276 that engage the protrusions (not shown) formed on an inner surface 128 of the mouthpiece 112 adjacent to the mouthpiece inlet 124. The threads 276 and mouthpiece protrusions cooperate to connect the carriage 214 to the mouthpiece 112. The threads 276 and mouthpiece protrusions are arranged so that the mouthpiece inlet end 112a abuts the shoulder 278 when the carriage 114 is connected to the mouthpiece 112. In addition, the outer surface of the insertion portion 274 includes flat regions 280 located between the threads 276 and the shoulder 278. When the cartridge 214 is connected to the mouthpiece 112 via the threads 276, a space is formed between the mouthpiece interior surface 128 and the flat regions 280, forming an air bypass port 284 that permits air flow into the mouthpiece fluid flow passage 116 (FIG. 19-21).

The housing second end 272 is open, defining a plunger-receiving port 286 through which the plunger 220 is inserted in the housing second end 272. The inner surface 266 of the housing 260 is provided with threads 282 that are configured to engage with corresponding threads 228 formed on an outer surface of the main plunger 228, as discussed below.

The housing sidewall 268 includes a sidewall opening 288 through which the cartridge 110 can be inserted into the reservoir 262. The sidewall opening 288 has a generally rectangular shape, and extends substantially along the full length of the sidewall 268 in an axial direction of the sidewall 268, and extends along about half the sidewall circumferential dimension in a circumferential direction of the sidewall 268.

The housing first end 270 is open, defining a carriage outlet 290 that is in fluid communication with the reservoir 262. The housing 260 includes a rigid awl 292 that is used to pierce a hole, or enlarge an existing hole (e.g., inlet hole 144), in an end of a cartridge 110 when the cartridge is disposed in the housing reservoir 262. The awl 292 is supported along the longitudinal axis 264 via a strut 298 that extends diagonally between opposed inner surfaces 266 of the reservoir 262. The strut 298 is positioned at an axial location corresponding to the shoulder 278, and the awl 292 protrudes axially from the strut 298 toward the housing second end 272. The awl 292 includes conical tip 296, and a shaft 294 that connects to the conical tip 296 to the strut 298. The conical tip 296 is enlarged relative to the shaft 294, and terminates in a sharp apex 297 at an end that is furthest from the strut 298.

The main plunger 220 is connected to the housing second end 272 and is a hollow cylindrical member including a main plunger sidewall 222 that extends between an open first end 230 and an open second end 232 that is opposed to the first end 230. The main plunger sidewall 222 defines an internal through passage 234 that receives and supports the spiked plunger 240. In some embodiments, the outer surface 224 of the main plunger has a diameter that corresponds to an inner diameter of the housing 260, is provided with threads 228 that engage with the corresponding threads 282 formed on the inner surface 266 of the second end 272 of the housing 260. The main plunger 220 is rotatably connected to the housing 260 via the cooperation of the threads 276, 282, and can be axially positioned relative to the housing 260 by rotating the main plunger 220 relative to the housing 260 about the housing longitudinal axis 264. In other embodiments, the main plunger may be connected to the housing 260 via other connection techniques including using a press fit, a magnetic retaining mechanism, a twist mechanism, a snap mechanism, a bayonet mount mechanism, or combinations thereof.

The main plunger sidewall 222 has an enlarged outer-diameter portion at the second end 232 forming an outwardly protruding flange 236 that can be easily grasped by the user's fingers to rotate the main plunger relative to the housing 260. In addition, the flange 236 has an outer diameter that corresponds to the outer diameter of the housing 260, whereby a shoulder 238 is formed between the flange and the main plunger sidewall 222. The shoulder 238 limits the extent to which the main plunger 220 can be axially moved relative to the carriage 114 toward the housing first end 270. An outer surface of the flange 236 is provided with knurls that facilitate manual gripping the main plunger 220.

The main plunger sidewall 222 has a reduced inner-diameter portion at the second end 232 forming an inwardly protruding annular lip 239. The annular lip 239 is engaged by retaining arms 250 formed on the spiked plunger 240 so as to form an axially slideable connection between the spiked plunger 240 and the main plunger 220.

The spiked plunger 240 is connected to the main plunger second end 232 and is a disc-shaped member including a first side 244, a second side 246 opposed to the first side 244, and a thickness corresponding to the distance between the first side 244 and the second side 246 that is small relative to its outer diameter. The outer diameter of the spiked plunger 240 corresponds to the outer diameter of the main plunger flange 236 and the outer diameter of the housing 260.

The spiked plunger 240 includes retention arms 250 that protrude from the first surface 244 in a direction normal to the first surface 244. The outward-facing surfaces of the free ends of the retention arms 250 include protrusions 252 that extend in a direction perpendicular to the retention arms 250. The retention arms 250 are equidistantly spaced along a circumference of a circle having a diameter that corresponds to an inner diameter of the lip 239 of the main plunger second end 232. By this arrangement, when the spiked plunger 240 is assembled with the main plunger 220, the retention arms 250 reside within the main plunger open second end 232 (e.g., within the inner diameter defined by the lip 239). The retention arms 250 extend in parallel with the housing longitudinal axis 264, and serve as guide rails along which the spiked plunger 240 can slide relative to the main plunger 220 during use. The protrusions 252 engage a surface of the lip 239 and thus serve to retain the spiked plunger 240 in connection with the main plunger 220 while permitting the axial translation of the spiked plunger 240 relative to the main plunger 220 along a distance corresponding to the distance between the retention arm protrusions 252 and the first side 244. In addition, carriage air inlet openings 259 are formed in the gaps between the retention arms 250 when the spiked plunger 224 is in the outward-most position.

The spiked plunger 240 includes elongate, rigid spikes 256 that are used to pierce holes in an end of a cartridge 10 when the cartridge 110 is disposed in the housing reservoir 262. The spikes 256 protrude from the first surface 244 in a direction normal to the first surface 244 and have free ends that terminate in a sharp tip 258. The spikes 256 are tapered, having a minimum cross sectional dimension at the tip 258 and a maximum cross-sectional dimension adjacent the spiked plunger first side 244. The spikes 256 are equidistantly spaced along a circumference of the circle in an alternating manner with respect to the retention arms 250. The cross-sectional dimension of the spikes 256 is less than the maximum cross-sectional dimension of the awl tip 296.

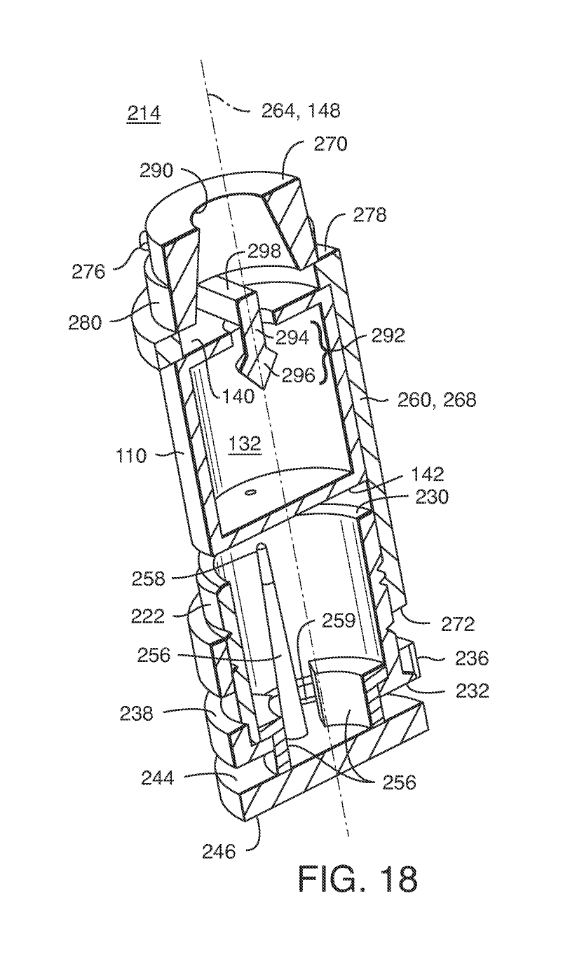

To prepare the carriage 214 for use, the carriage 214 is connected to the mouthpiece 112 forming the air delivery device 100, and then is configured to receive a cartridge 110 (e.g., placed in the “loading configuration”) (FIG. 19). In the loading configuration, the main plunger 220 is rotated to a position relative to the housing 260 that permits the cartridge 110 to be inserted through the sidewall opening 288 and into the reservoir 262. For example, the main plunger 220 may be rotated to a position in which the main plunger first end 230 is located between the housing second end 272 and the sidewall opening 288. In addition, the spiked plunger 240 is positioned so as to be in its axially outward-most position relative to the main plunger 220 (e.g., the spiked plunger is moved outward axially until the retention arm protrusions 254 abut the lip 239 formed on the inner surface of the main plunger 220). In this position, the spike tips 258 reside within the through passage 234 of the main plunger. When the carriage 214 is in the loading configuration, the cartridge 110 is inserted through the sidewall opening 288 into the housing reservoir 262 in a manner so that the cartridge longitudinal axis 148 is parallel to the housing longitudinal axis 264, the cartridge first end 140 faces the carriage housing first end 270, and the cartridge second end 142 rests on the main plunger first end 230 (FIG. 18).

After the cartridge 110 is placed inside the carriage reservoir 262, the carriage 214 is configured to provide air inlet and outlet openings in the cartridge 110 if none previously exist, to pierce seals that may cover pre-existing air inlet and outlet openings in the cartridge 110, or to ensure that previously formed air inlet and outlet openings are adequately sized (e.g., placed in the piercing configuration) (FIG. 20). In the piercing configuration, the main plunger 220 is rotated so as to advance the cartridge 110 axially toward the awl 292 to an extent that the awl tip 296 passes through the cartridge first end 140 forming (or enlarging) an air inlet opening 144 in the cartridge first end 140 having a dimension corresponding to the maximum dimension of the awl tip 296. In this configuration, the awl tip 296 resides within the cartridge 110, and the awl shaft 294 resides within the air inlet opening 144. In addition, the spiked plunger 240 is moved axially inward relative to the main plunger 220 to an extent that the first side 244 of the spiked plunger 240 abuts the main plunger second end 232. By doing so, the spikes 256 are advanced axially inward to an extent that the spike tips 258 pierce the cartridge second end 142, forming (or enlarging) air inlet openings 146 in the cartridge second end 142.

After the cartridge 110 has been pierced to form an air outlet opening and air inlet openings 144, 146, the carriage 214 is configured to be used (e.g., placed in the “use configuration”) (FIG. 21). In the use configuration, the spiked plunger 240 is moved axially outward relative to the main plunger 220 so as to be in its axially outward-most position relative to the main plunger 220, whereby the spikes 256 are withdrawn from the cartridge 110, and the cartridge air inlet openings 259 are formed between the retention arms 250. This position permits air to flow freely into the cartridge 110 via the cartridge inlet openings 146.

In the use configuration, the mouthpiece 112, the carriage 214 and the cartridge 110 together define a flow path through the device 100. Thus, when a user places the outlet 122 of the mouthpiece 112 in his or her mouth and inhales, air flows into the cartridge reservoir 132 through the cartridge inlets 146. Air then flows from the cartridge reservoir 132 through the cartridge air outlet 144, through the carriage outlet opening 290 and into the inlet 124 of the mouthpiece 112. Air is drawn into the mouthpiece 112 through the mouthpiece inlet 124. The air flows along the fluid flow passage 116, and exits the mouthpiece 112 via the mouthpiece outlet 122. Contact with the airflow directing member 118 deflects the air flowing out of the mouthpiece 112, redirecting it in a direction that is angled relative to a longitudinal axis 126 of the mouthpiece 112. For example, in the illustrated embodiment, the airflow directing member 118 redirects the air flowing out of the mouthpiece 112 in a direction that is substantially perpendicular to a longitudinal axis 126 of the mouthpiece 112, but the deflection angle can be greater or lesser than 90 degrees.

A user operates a particle delivery device 100 by loading the device 100 (e.g., placing a cartridge 110 in the reservoir 262 of the carriage 214 (the carriage 214 can be connected to the mouthpiece either before or after loading the cartridge into the carriage 114), bringing the device 100 to the user's mouth, and inhaling through the mouthpiece 112 thereby causing air to enter the carriage 214 and mouthpiece 112 through the air passageways. The air aerosolizes the powder present in the cartridge reservoir 132. The aerosolized powder subsequently enters the user's mouth via the mouthpiece 112.

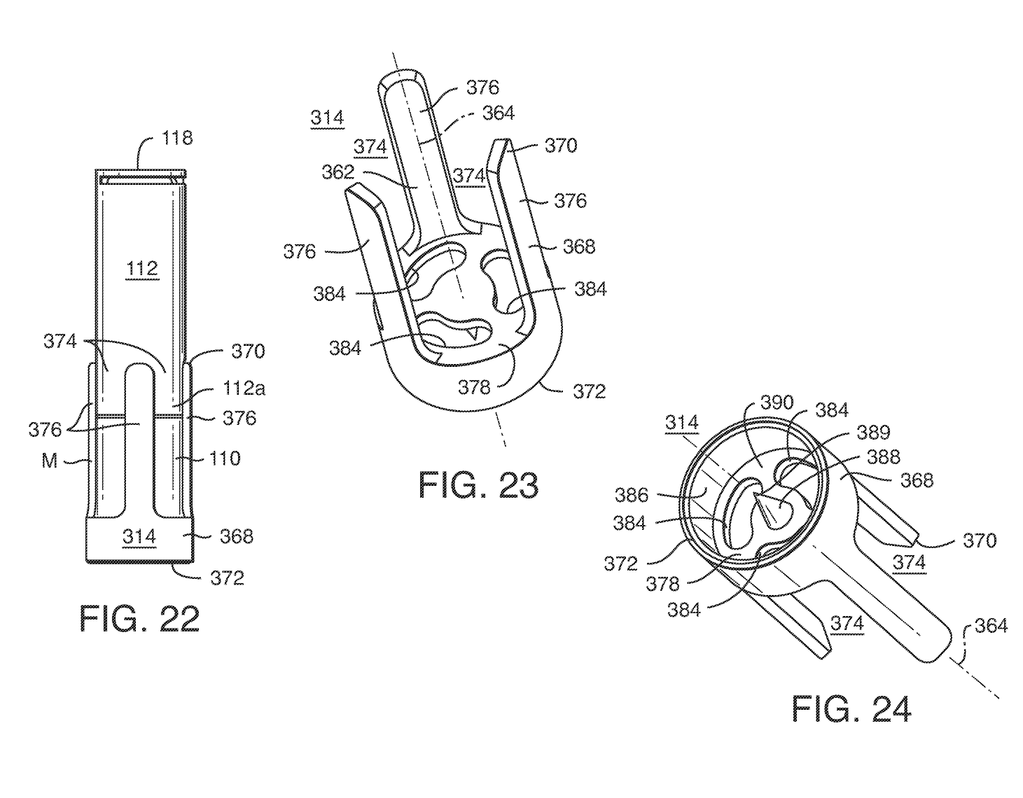

Referring to FIGS. 22-24, another carriage 314 can be used with the mouthpiece 112. The carriage 314 is a hollow cylindrical member including an annular sidewall 368, an open first end 370 and an open second end 372 opposed to the first end 370. A longitudinal axis 364 of the carriage 314 extends through the respective centers of the first and second ends 370, 372. The sidewall 368 has a cross-sectional shape and an inner dimension that corresponds to the cross-sectional shape and outer dimension of the mouthpiece 112. The carriage 314 includes a partition 378 that extends transverse to the cartridge longitudinal axis 364. The partition 378 includes elongated through openings 384 that are equidistantly spaced apart from each other. Each through opening 384 is disposed between a center of the partition 378 and the sidewall 368. The partition 378 separates the inner space defined by the sidewall 368 into a reservoir portion 362 and a piercing portion 386. The reservoir portion 362 and piercing portion 386 are in fluid communication via the through openings 384. The partition 378 is located between the mid-portion M of the carriage 314 and the carriage second end 372 so that the reservoir portion 362 is much larger (e.g., has a greater volume) than the piercing portion 386.

The first end 370 of the sidewall 368 includes three U-shaped cut out regions 374 that extend axially inward between the first end 370 and the partition 378. The portions 376 of the sidewall 368 intermediate the cut out regions 374 (e.g., the legs 376) serve to connect the carriage 314 to an outside surface of the inlet end 112a of the mouthpiece 112. To that end, the legs 376 have an inner dimension that corresponds to an outer dimension of the mouthpiece 112. In addition, the legs 376 have an axial dimension that is longer than the axial dimension of the cartridge 110. In some implementations, the legs 376 have a length that is in a range of 15 to 30 percent longer than the axial dimension of the cartridge 110.

The carriage 314 includes a conical spike 388 disposed in the piercing portion 386. The spike 388 protrudes from the piercing portion-facing surface 390 of the partition 378 in a direction normal to the partition surface 390, and is tapered to a sharp tip 389. The spike 388 has a length (e.g. distance from partition surface 390 to the tip 389) that corresponds to the axial length of the piercing portion 386.

In use, the carriage 314 serves as both a piercing tool and a component that holds a cartridge 110 in position at an inlet of the mouthpiece 112. For example, prior to connecting the carriage 314 to the mouthpiece 112, the carriage 314 is used as a piercing tool by pressing the cartridge 110 within the piercing portion 386 to an extent that the first end 140 of the cartridge 110 is pierced by the spike 388 forming an air inlet opening 144 in the cartridge first end 140. After forming the air inlet opening 144, the cartridge 110 is removed from the piercing portion 386. The carriage 314 is then used as a holder by placing the cartridge 110 into the reservoir portion 362 with the cartridge second end 142 abutting the partition 378. It is understood that the partition openings 384 provide free air flow to the inlet openings 146 of the second end 142 of the cartridge 110, and are elongated so as to facilitate alignment with the openings 146. Once the cartridge 110 is loaded into the reservoir portion 326, the carriage is connected to the mouthpiece 112 by sliding the legs 376 over an outside surface of the mouthpiece 112 until the cartridge first end 140 abuts the mouthpiece inlet end 112a. The carriage 314 is retained on the mouthpiece 112 by frictional engagement between an inside surface of the legs 376 and the outside surface of the mouthpiece 112.

Here, the carriage 314 is not limited to being mounted to the outside of the mouthpiece 112, and could alternatively be mounted to an inside surface of the mouthpiece 112 by appropriate dimensioning of outer diameter of sidewall 368 in the vicinity of the legs 376.

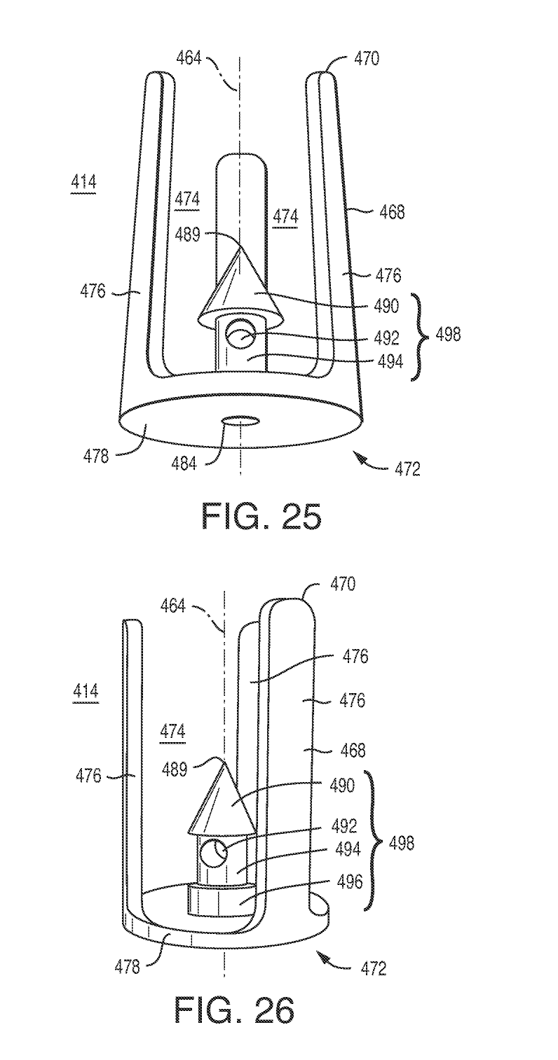

Referring to FIGS. 25-26, another carriage 414 can be used with the mouthpiece 112. The carriage 414 is a hollow cylindrical member including an annular sidewall 468, an open first end 470 and a second end 472 opposed to the first end 470. A longitudinal axis 464 of the carriage 414 extends through the respective centers of the first and second ends 470, 472. The sidewall 468 has a cross-sectional shape and an inner dimension that corresponds to the cross-sectional shape and outer dimension of the mouthpiece 112. The carriage 414 includes a base 478 at the second end 472 that extends transverse to the cartridge longitudinal axis 464. The base 478 includes an opening 484 located in the center of and extending through the base 472 and oriented along the longitudinal axis 464.

The first end 470 of the sidewall 468 includes three U-shaped cut out regions 474 that extend axially inward between the first end 470 and the second end 472. The portions 476 of the sidewall 468 intermediate the cut out regions 474 (e.g., the legs 476) serve to connect the carriage 414 to an outside surface of the inlet end 112a of the mouthpiece 112. To that end, the legs 476 have an inner dimension that corresponds to an outer dimension of the mouthpiece 112. In addition, the legs 476 have an axial dimension that is longer than the axial dimension of the cartridge 110. In some implementations, the legs 476 have a length that is in a range of 15 to 30 percent longer than the axial dimension of the cartridge 110.

The carriage 414 includes a conical spike 490 located on a shaft 494. The shaft 494 is located on a shaft base 496. The conical spike 490, the shaft 494 and the shaft base 496 comprise the piercing assembly 498. The piercing assembly 498 is centrally disposed along the longitudinal axis 464, with the shaft base located on the opening 484 of the second end 472 and oriented with the conical spike 490 proximal to the open first end 470. The piercing assemble 498 is hollow. At least one air exit hole 492 extends through the shaft 494, and the air exit hole 492 is in fluid communication with the opening 484. The spike 490 protrudes in a direction normal to the base 478, and is tapered to a sharp tip 489. The piercing assembly 498 has a length (e.g. distance from the base 478 to the tip 489) that corresponds to a distance sufficient for the conical spike 490 to extend through an edible cartridge base 142 and into a powdered payload within the edible cartridge 130. The widest dimension of the conical spike 490 is approximately the dimensional width of the shaft base 496.

In use, the carriage 414 simultaneously serves as both a piercing tool and a component that holds a cartridge 110 in position at an inlet of the mouthpiece 112. For example, as an edible cartridge 130 is pressed onto the conical spike 490, the edible cartridge base 142 is pierced by the conical spike 490 and then pressed towards the shaft base 496 where the wall of the pierced-hole inner dimension will tolerance fit to the outer dimension of the shaft base to form a seal. After forming or unsealing an air inlet opening 144, the carriage 414 is used as a holder and placed in contact with the mouthpiece 112 by sliding the legs 476 over an outside surface of the mouthpiece 112 until the cartridge first end 140 abuts the mouthpiece inlet end 112a. It is understood that the opening 484 is in fluid communication with the air exit hole 492 to provide free air flow to the newly formed inlet openings of the second end 142 of the cartridge 110. The carriage 314 is retained on the mouthpiece 112 by frictional engagement between an inside surface of the legs 376 and the outside surface of the mouthpiece 112.

Here, the carriage 414 is not limited to being mounted to the outside of the mouthpiece 112, and could alternatively be mounted to an inside surface of the mouthpiece 112 by appropriate dimensioning of outer diameter of sidewall 468 in the vicinity of the legs 476.

In the embodiment illustrated in FIGS. 1-9, the carriage 114 is connected to the mouthpiece via engagement between carriage grooves 180 and corresponding mouthpiece protrusions, but the carriages 114, 214, 314 disclosed herein are not limited to this type of connection. Various strategies can be employed for attaching the reusable and/or replaceable carriages 114, 214, 314 described herein to the mouthpiece 112. In some embodiments, a first end 170, 270, 370 of the carriage 114, 214, 314 has an outer surface that is sized and configured to provide a snap-fit engagement with the inner surface of the corresponding end of the mouthpiece 112. In some embodiments, other forms of engagement are used instead of or in addition to snap-fit engagement to attach the carriage 114, 214, 314 to the mouthpiece 112. In certain configurations, the carriage 114, 214, 314 is held or locked into position through mechanical tension and/or frictional forces from the particular mounting design. For example, in some embodiments, the carriage 114, 214, 314 and the mouthpiece 112 have threads and are screwed together (see for example FIGS. 13-21). In another example, carriages are slidably and reversibly interference or press fit pressed into position within the inlet end 112a of the mouthpiece 112. In some embodiments, the inlet end 112a of the mouthpiece 112 is of a slightly larger diameter than the diameter of the corresponding mating end of the carriage 114, 214, 314, creating a press-fit configuration between the pieces. In other embodiments, the mouthpiece and carriage are locked into position with spring tension locating pins, dowels, ball bearings, living hinge positioners, etc. In still other embodiments, the carriage 114, 214, 314 is held into place with the mouthpiece via magnetic holders and a ferrous and/or magnetic counterpiece. Other alternative structures for connecting the carriage 114 to the mouthpiece 112 embodied in the present invention include, but are not limited to, a screw fit, twist fit, snap fit, press fit and turn, bayonet mount, etc. In still other embodiments, the mouthpiece and carriage are fixed to each other, forming an integral unit.

Although the carriage 114 has been disclosed as including a door 190 that pivots on pins 192 about the pin axis 194, the door 190 is not limited to this configuration. For example, in some embodiments, the door 190 is connected to the sidewall 168 via a hinge such as a living hinge so as to pivot about the pin axis 194. For example, in other embodiments, the door 190 pivots about an axis transverse to the pin axis 194. In still other embodiments, the door 190 does not pivot, and instead is tethered to the sidewall 168 so as to be completely removable from the sidewall 168 while remaining attached to the carriage 114.

In some embodiments, the carriage 114, 214, 314 is formed of a resilient material.

In some embodiments, the components constituting the carriage 114, 214, 314 are manufactured from a plastic. In certain embodiments, the plastic is biodegradable. In other certain embodiments, the components constituting the carriage 114, 214, 314 are manufactured from a polyester, a polyhydroxyalkanoate, a polyanhydride, a polycaprolactone, a polydiaxonone, a polyglycolide, a polyhydroxybutyrate, a polylactic acid, a polypropylene carbonate, a polylactic-co-glycolic acid, a poly(3-hydroxybutyrate-co-3-hydroxyvalerate, a polyvinyl alcohol, a starch derivative, cellulose esters, a cellophane, an enhanced biodegradable plastic, compositional variants thereof, combinations thereof, etc.

In certain embodiments the cartridge 110 is edible and manufactured from a starch, a grain-based food, a vegetable, a meat, a fruit, a dairy product, a sugary food, a nut, a confection, a plant product, processed edible products thereof, synthetic edible products thereof, combinations of edible products, etc.

In certain embodiments the cartridge 110 is edible or biodegradable, and manufactured from chocolate, bread, fruit, sugar, meat, bread, pasta, processed forms thereof, combinations thereof, etc.

In some embodiments, the body of the entire device 100 is manufactured from a starch, a grain-based food, a vegetable, a meat, a fruit, a dairy product, a sugary food, a nut, a confection, a plant product, processed edible products thereof, synthetic edible products thereof, combinations of edible products, etc.

In some embodiments, the device 100 may be similar to an inhaler or inhalation device, such as a dry powder inhaler (DPI) or metered dose inhaler (MDI); a “pot” that holds an ultrasound source and confines somewhat the aerosol cloud produced by the source; a “fountain” that ejects and/or circulates the aerosol; a hand-held pump device; a compressed air device; a food straw device; a multi-person, communal device; a tabletop device. A variety of materials may be used to form the device, or parts thereof, including: plastics (e.g. polycarbonates, which are relatively strong, polypropylene, acrylonitrile butadiene styrene, polyethylene, etc.), various metals, glass, cardboard, rigid paper, etc.

In certain embodiments, the aerosolizing device 100 includes a disposable and/or replaceable cartridge 110. For example, the reusable or replaceable cartridge is selectively detachable from the device 100 and has a volume separate from the device 100 and includes an inlet and/or outlet port that can be closed or sealed. This is advantageous in the event that not all the payload has been delivered, since the device can be selectively placed in a closed configuration until further usage at a later time. In addition, a closable or sealable device is advantageous in situations wherein the user exchanges one cartridge for another cartridge without having expended all payload in the first cartridge. The partially used, sealed first cartridge can be reconnected to the mouthpiece at a later time for further use.

The cartridge 110 may not be readily opened to access the consumable product inside. This is advantageous for instances in which it is preferable that the user have limited direct access to the consumable product (i.e., access other than by delivery via the mouthpiece as intended). This may be the case when the consumable product is a controlled substance, a sensitive substance, or a substance that requires a very precise dose. This may also reduce the risk of a user inserting other materials into the device.

In some embodiments, the cartridge 110 is an assembly of multiple components 134, 136 that together define concave inner spaces 132, and, after powder is filled into either or all of the components, the components snap or screw together to form a largely closed interior chamber. In some embodiments, the cartridge 110 further include an aerosol generating device, for example, an airflow-disrupting “grating” (not shown), through which air and powder flow, thereby yielding an aerosol for delivery to the user. The cartridge typically includes air passageways, for example, on the respective ends of the enclosed compartments, so as to allow air to flow through upon inhalation. The design, for example, the size or shape of the air passageways, should provide sufficient airflow while minimizing powder loss.

In the illustrated embodiments, the cartridge 110 includes upper element 134 and lower element 136. While a consumer is not able to readily separate the upper and lower elements, the two elements may initially separate so as to permit filling of the cartridge 110 during the manufacture of the overall product. In this embodiment, the housing 130 is filled with the consumable product during the manufacture of the overall product, and shortly thereafter, the cover or cover assembly is affixed to the housing, for example by a snap fit. The snap fit can be designed to resist being taken apart once the two elements are affixed. In some embodiments, other methods of attachment such as, for example, press fits and ultrasonic welding can be used to attach the cover and the housing.

In some embodiments, the device 100 is designed for single use (perhaps disposable) or, alternatively, designed for multiple use. For example, in some embodiments, the cartridge 110 may be disposable, and, optionally, available with a variety of food powders, while the mouthpiece and cartridge 114, 214, 314 may be reusable. In certain embodiments, pre-filled standard-sized capsules, for example, a gel capsule, blister pack, or sealed capsule of another form, can be used by placing them in the cartridge reservoir 132. Such embodiments allow for easier filling, substitution, cleaning, and disposal. In addition, such embodiments allow for manufacture of multiple dose capsules. Such pre-filled capsules could be punctured, torn, cut or broken by design elements within the housing (for example, sharp points, blades, compressing the device, or twisting the device etc.) prior to use. In some cases, a sealing member is removed; for example, a sealing plastic or metal foil initially adhering to the capsule (or cap) can be peeled off. In some cases, the capsule can be protected or sealed with a cover. The product may thus be released into the reservoir 132, for example, and become more susceptible to airflow generated during inhalation or activation. In another embodiment, the aerosolizable product may remain substantially within the original container but now be in fluid communication with, and thus now susceptible to, airflow generated during inhalation and/or activation, etc. After activation and use, the emptied capsule could be removed from the reservoir 132. Alternatively, the cartridge can be designed for multiple uses. For example, the cartridge may be refillable. In some cases, the cartridge may be designed for one or more uses, and not be itself refillable. In some cases, used (e.g., empty) cartridge can be readily removed from contact, or removed from fluid communication, with the mouthpiece 112. In some cases, new (e.g., filled) cartridge can be readily brought into contact or fluid communication with the mouthpiece 112. In some cases, such fluid communication can be achieved using clean or sterile components.

In some embodiments, a cartridge can contain more than one dose. For example, in some embodiments, multiple doses are contained within a single larger cartridge, in which different doses are physically separated from each other. In some embodiments, a mouthpiece 112 is in fluid communication with only one of these doses at a given time during use. Once the dose is delivered, it is possible for the mouthpiece 112 to be put in fluid communication with a different dose container within the cartridge, containing a new dose of consumable product. As such, a single mouthpiece 112, and a single cartridge 114, can be used to deliver multiple doses. After consuming all doses within a capsule element, the capsule element may be replaced or refilled.

In the illustrated embodiments, the mouthpiece 112 is formed having a circular or oval cross section, but it is not limited to these cross-sectional shapes. For example, the mouthpiece can have a square or rectangular cross-sectional shape.

In the illustrated embodiment, the mouthpiece 112 is a monolithic structure (e.g., formed of a single piece). However, in other embodiments, the mouthpiece 112 can be an assembly of at least two substructures. For example, the mouthpiece can be an assembly of an end piece with a deflection member designed for oral placement and a body that connects the mouthpiece to a consumable particle cartridge and/or capsule. In another example, the mouthpiece can be an assembly of three separate components including an end piece for oral placement, a deflector which may or may not be detachable from the end piece, and a body that connects the end piece to a consumable particle cartridge and/or capsule. Other configurations of a mouthpiece with equivalent function to that as described herein are contemplated. Other mouthpieces can be used with aerosolized particle delivery devices.

In some embodiments, the mouthpiece 112 is manufactured from a plastic. In certain embodiments, the plastic is biodegradable. In other certain embodiments, the mouthpiece 112 is manufactured from a polyester, a polyhydroxyalkanoate, a polyanhydride, a polycaprolactone, a polydiaxonone, a polyglycolide, a polyhydroxybutyrate, a polylactic acid, a polypropylene carbonate, a polylactic-co-glycolic acid, a poly(3-hydroxybutyrate-co-3-hydroxyvalerate, a polyvinyl alcohol, a starch derivative, cellulose esters, a cellophane, an enhanced biodegradable plastic, compositional variants thereof, combinations thereof, etc.

In some implementations, the mouthpiece 112 is intended to be reused, for example, for 2, 5, 10, 50, 100, or more deliveries of consumable product. In certain embodiments, reusable parts of the device can be used indefinitely (e.g., they can be readily cleaned, in a dishwasher, by hand, etc.). For example, a mouthpiece made of a durable and cleanable material, such as certain metals (i.e., stainless steel), plastics, ceramic or glass, may be used in conjunction with many cartridges over time.

In other implementations, the mouthpiece 112 can be re-used, for example, with multiple carriages of the same type (e.g., of the same embodiment), or in another example, with carriages of different types (e.g. of a different embodiment).

In other implementations, the other mouthpieces may be used with the carriage 114, 214, 314.

In the embodiments described above, the mouthpiece 112 and carriage 114 are formed of durable, washable and/or sanitizable materials such as aluminum, stainless steel or plastic, while the cartridge is formed of edible materials. However, the device 100 is not limited to this configuration. For example, in some embodiments, the body of the entire device 100 is manufactured from a starch, a grain-based food, a vegetable, a meat, a fruit, a dairy product, a sugary food, a nut, a confection, a plant product, processed edible products thereof, synthetic edible products thereof, combinations of edible products, etc.

In many instances, variations of some embodiments may be designed without, in many instances, affecting the function of the overall device. For example, the cylindrical nature of the device may be modified, for example, for aesthetic effect, as may the overall length of the device.

In some embodiments, the cartridge 110 is mounted to a carriage 114, 214, 314 (e.g., received within a reservoir of the carriage), and the carriage is then mounted to the mouthpiece 112. For example, the cartridge 110 can be placed into a mounting carriage, with the carriage designed to accommodate cartridge geometry and align carriage ports with inlet and/or outlet locations on the cartridge. Dowels, pins or other locating devices can then mount the carriage/cartridge to the mouthpiece or first member in the appropriate orientation.

In the described embodiments, the cartridge 110 is mounted to the mouthpiece 112 using the carriage 114, 214, 314. However, in some embodiments, the cartridge 110 can be directly secured to the mouthpiece for example via by detachably press fitting the cartridge 110 into the mouthpiece 112, or alternatively abutted to the end of the mouthpiece 112.

### Activation of Aerosolization and Delivery of Consumable Product

By controlling gravitational and inertial forces, the airflow-directing elements found in some embodiments enable delivery of the aerosol cloud substantially to surfaces within the mouth (i.e., tongue, cheeks, etc.) rather than into the respiratory tract. This aspect of the technology is highly relevant to a number of potential applications of aerosolizable products. Indeed the same delivery device can make possible delivery of a wide range of aerosol products, generated in a number of different ways, to a consumer, while minimizing or eliminating coughing and potential interactions with surfaces of the respiratory system beyond the mouth.

The design of any of the devices and formulations described herein is configured for the reduction of the tendency to cough, gag, or otherwise react unfavorably to an aerosolized product, and can be embodied in various configurations and formulations to achieve that purpose; the devices and formulations described herein are meant to be exemplary.

Air flow of the device is moderated for user comfort and aerosolizing capacity of the device. Optimal airflow can be dependent on factors including payload, device design, air inlet port size and configuration, air bypass size and configuration, and overall size of the device. Generally, the device is designed for hand held use. In certain embodiments the aerosolizing delivery device is configured to permit a flow rate through the device of between about five (5) liters per minute and about sixty (60) liters per minute at a vacuum pressure of about four kiloPascals. In certain embodiments the aerosolizing delivery device is configured for a flow rate through the device of between about ten (10) liters per minute and about thirty (30) liters per minute at a vacuum pressure of about four kiloPascals. In certain embodiments the aerosolizing delivery device is configured for a flow rate through the device of between about fifteen (15) liters per minute and about twenty-five (25) liters per minute at a vacuum pressure of about four kiloPascals. It may also be desirable to achieve flow rates that deliver a portion, or all of, the powder contained in the cartridge reservoir or volume in one, two, three, four, five, six, seven, eight, none, ten or more actuations, wherein an actuation is about a one to two second inhalation at about 4 kiloPascals of applied vacuum pressure. In certain embodiments, the payload of delivered powder per actuation at 4 kiloPascals applied vacuum pressure for about 1 to 2 seconds of inhalation, is between live and 10 percent of the starting quantity of powder contained in the cartridge reservoir. In certain embodiments, the payload of delivered powder per actuation at 4 kiloPascals applied vacuum pressure for about 1 to 2 seconds of inhalation, is between ten and twenty percent of the starting quantity of powder contained in the cartridge reservoir. In certain embodiments, the payload of delivered powder per actuation at 4 kiloPascals applied vacuum pressure for about 1 to 2 seconds of inhalation, is between twenty and thirty percent of the starting quantity of powder contained in the cartridge reservoir. In certain embodiments, the payload of delivered powder per actuation at 4 kiloPascals applied vacuum pressure for about 1 to 2 seconds of inhalation, is between thirty and forty percent of the starting quantity of powder contained in the cartridge reservoir. In certain embodiments, the payload of delivered powder per actuation at 4 kiloPascals applied vacuum pressure for about 1 to 2 seconds of inhalation, is between forty and fifty percent of the starting quantity of powder contained in the cartridge reservoir. In certain embodiments, the payload of delivered powder per actuation at 4 kiloPascals applied vacuum pressure for about 1 to 2 seconds of inhalation, is between fifty and sixty percent of the starting quantity of powder contained in the cartridge reservoir. In certain embodiments, the payload of delivered powder per actuation at 4 kiloPascals applied vacuum pressure for about 1 to 2 seconds of inhalation, is between sixty and seventy percent of the starting quantity of powder contained in the cartridge reservoir. In certain embodiments, the payload of delivered powder per actuation at 4 kiloPascals applied vacuum pressure for about 1 to 2 seconds of inhalation, is between seventy and eighty percent of the starting quantity of powder contained in the cartridge reservoir. In certain embodiments, the payload of delivered powder per actuation at 4 kiloPascals applied vacuum pressure for about 1 to 2 seconds of inhalation, is between eighty and ninety percent of the starting quantity of powder contained in the cartridge reservoir. In certain embodiments, the payload of delivered powder per actuation at 4 kiloPascals applied vacuum pressure for about 1 to 2 seconds of inhalation, is between ninety and one hundred percent of the starting quantity of powder contained in the cartridge reservoir.

All references to a powder, liquid, aerosol, cloud, particle, etc. made herein may equivalently refer to some fraction or portion of the total amount of the powder, liquid, aerosol, cloud, etc.

In some embodiments, the aerosolized product should be of a determined size, i.e., of sufficient size to limit entry into the respiratory tract but of small enough size to allow for suspension in the air. In some embodiments, particle size may be a manufacturing requirement of pre-atomized, generally solid products, for example the products placed inside the capsule/cap of certain embodiments, or certain dry products used in association with an air pump or compressed air source. In some embodiments, particle size may be a requirement of the aerosol-generating device, for (generally liquid) products that are only atomized upon aerosol generation, for example the products used in association with ultrasound sources to produce an aerosol cloud.

In some embodiments, the predetermined, mean size of the aerosolized product is at least 1, 2, 3, 4, 5, 10, 15, 20, 25, 30, 35, 40, 45, 50, 55, 60, 70, 75, 80, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 325, 350, 375, 400, 425, 450, 475, or 500 microns. In some embodiments, the predetermined, mean size of the aerosolized product is less than 500, 450, 400, 350, 325, 300, 275, 250, 245, 240, 235, 230, 225, 220, 215, 210, 205, 200, 195, 190, 185, 180, 175, 150, 140, 130, 120, 110, 100, 90, 80, 70, 60, 50, 40, 30, 20, or 10 microns in size. Ranges intermediate to those recited above, e.g., about 50 microns to about 215 microns, are also intended to be part of this disclosure. For example, ranges of values using a combination of any of the above recited values as upper and/or lower limits are intended to be included.

Especially, but not exclusively, in some embodiments in which intake is by inspiration or expiration, minimum particle size is an important feature of the approach. The food aerosol particles are designed to be substantially delivered and deposited into the mouth, for example by the forces of gravity or inertial impaction, but to not be easily delivered and deposited substantially further into the respiratory tract, for example the trachea or lungs. Such consumable particles would thus possess a size larger than that which focuses penetration into the lungs (i.e., larger than about 10 microns). For example, breath-activated aerosolizing devices, such as the devices shown (in part or in whole) herein, generate an aerosol that would fairly easily follow the inspired air toward the lungs were it not for the aerosol particles' larger size (and the delivery device's airflow-directing elements).

Especially, but not exclusively, in embodiments in which intake is by displacement of the subject or of the aerosol (e.g., with an aerosol cloud), maximum particle size is an important feature of the approach. Indeed, the aerosol cloud must remain suspended in air for at least a brief time so that displacement into the mouth can occur. Thus the particles must not be so large such that they rapidly settle from the air. This will greatly depend on the force(s) and/or mechanism(s) by which the particles are held in the air (e.g., by “natural” forces alone, such as inertia, diffusion, etc., or by additional forces, such as an impeller, air currents, convection, etc.). Accordingly, in some embodiments, the particles should be less than about 500 microns under typical suspension forces and mechanisms. For example, ultrasound sources in liquid products can produce a standing aerosol cloud that, so long as convection is minimal, balances gravity, diffusion, inertial impaction, and other forces, to stay suspended in the air.

The specific parameters of the device and intake method will in part determine whether the subject is inhaling/exhaling or eating/sipping when intake of the aerosol occurs. This generally corresponds to (1) whether the aerosol is entering the subject's mouth and/or throat via breathed air (physiologically, while the epiglottis is directing the air into the trachea toward the lungs) or whether the aerosol is entering the subject's mouth by another method (such as displacement of the aerosol or of the subject), and (2) whether the subject's maneuver or expectation is equivalent to the consumption of a food product to be (eventually) swallowed (e.g., as with the use of a drinking straw while drawing fluid into the mouth, before swallowing; physiologically, while the epiglottis is blocking passage to the trachea). In any case, it should be further noted that the product, after deposition in the mouth, may be eventually swallowed and consumed essentially as any other typical ingestible product.

In some cases, the aerosol may be carried via inhaled air that flows all the way to the lungs, for example, like the inhalation a smoker may have, which carries air and smoke through/from the cigarette, into the lungs. In some cases, the aerosol may be carried via sucked air that stops in the mouth, more like the approach used with a typical straw and beverage, or with cigars. In some cases, elements of both approaches may be suitable. This potential distinction may have important implications for an aerosolizing device. For example, in the case in which the particles are carried by air that continues directly to the lungs, preventing deposition of particles too far into the respiratory tract is more dependent on the physical parameters of the particles, airflow, etc. In the case in which the particles are carried by air that is sucked into the mouth, it may be possible to carry particles of mean sizes, or with other properties, that would normally allow them to extend further than desirable into the respiratory system, but that, by virtue of the airflow stopping before the lungs, have them fall substantially into the mouth anyway.

In some embodiments of devices in which an aerosol is generated by inhalation, relatively dry, solid powders of appropriate size can be used as the product. Preliminary tests have shown that the water-solubility of the dry powders used plays a role in the taste and potential coughing reflex resulting from intake of the aerosolized product. For example, powders of particles that tend to be more rapidly water-soluble, such as ground chocolate bars, or certain chocolate-based powders, give rise to a generally pleasing reaction upon contact of the particles with the tongue and other surfaces within the mouth. In the case of ground chocolate bars, for example, the effect is in some cases similar to that of sensing chocolate melt very rapidly in one's mouth. Conversely, particles that are less water-soluble, such as certain ground-cocoa-based powder products, tend to be considered harsher and more likely to elicit less pleasurable reactions, such as a dry-mouth sensation or coughing. However, in some instances, a combination of both kinds of powders, in varying proportions, provides interesting flavor complexity.

### Consumable Aerosol Powders and Edible Powder Cartridges

Dry powder particles for use as a payload and as components of edible cartridges can be created through a number of different methods. Initially, the ingredients of a formulation may be dehydrated. In some embodiments, where the ingredient is a more malleable or liquid based food, the ingredient may be frozen first to facilitate subsequent grinding or chopping. The ingredient may subsequently be ground to form particle products of the appropriate size. Grinding of the products can be performed by use of a mortar and pestle. Alternatively or in addition, products may be chopped, for example using a mechanical or electrical grinder, knives, etc. The resulting ground or chopped particles can subsequently be filtered through sieves (for example by hand, using an electrical or mechanical sieve shaker, by an air classification system, by a screening system, etc.) to achieve the appropriate particle size.

In some embodiments, a powder mill grinds down larger particles into pre-defined sizes.

In certain embodiments, spray drying, in which a mixture of water and the material to be dried is forced through a nozzle into a high-temperature drum, instantly evaporating the water droplets clinging to the material, may be utilized. Spray drying gives the most consistent desired particle size distribution and population density, increasing the output of particles having the desired performance characteristics.

These methods, in addition to others, would allow for the creation of specifically sized particles capable of being aerosolized, but large enough not to pass easily through the mouth and throat and continue into the respiratory tract.

By designing a dry powder formulation that can be aerosolized (particles much larger than 500 microns fall quickly out of the air unless supported by an external force) and yet has sufficiently large particles (greater than approximately 10, 15 or 20 microns) such that few or no particles enter the lungs on inspiration, our technology results in deposition and delivery into the mouth. Ideally, the particles would be designed (sized) such that, for example, at least about 50%, at least about 60%, at least about 70%, at least about 80%, at least about 90%, at least about 95%, at least about 97%, or at least about 99% of the particles deposit in the mouth and do not extend further into the respiratory tract. The design of the particles should also take into consideration reducing any tendency to cough, gag, or otherwise react unfavorably to the ingestible powder when aerosolized.

In the preparation of consumable, aerosolizable products (e.g. food powders, dietary supplements, pharmaceuticals, etc.) intended for use with aerosol delivery mechanisms as described herein, it may be desirable to modify the particle size distribution. In some cases, it may be desirable to increase the overall mean particle size of an aerosolizable, consumable product. In some cases, it may be desirable to increase or decrease the number (or mass) of particles of a first size range (representing a subset of the overall initial size distribution), relative to the number (or mass) of particles of a second size range (representing a subset of the overall initial size distribution).

Specifically, in some embodiments, one may wish to significantly reduce the number/mass of “finer” particles relative to the number/mass of “larger” particles. Reducing the number/mass of finer particles is particularly relevant to applications in which an aerosol is brought into the body via the mouth, and in which it is intended that aerosol particles deposit substantially on surfaces of the mouth before reaching the back of the throat or further into the respiratory tract. This is particularly true when the original size distribution includes a substantial proportion of particles of a size that can be conducive to respiratory penetration (e.g., particles of a size less than about 2, 5 or 10 microns).

In some embodiments, changing a consumable, aerosolizable product from a first size distribution to a second size distribution is done in a way that preserves (or enhances) useful or desirable qualities. For example, the water-solubility of a consumable aerosol product may impact its dissolution within the mouth (e.g., into saliva), the taste it elicits, the risk of coughing, etc. Changing the particle size distribution should be done using a process that does not substantially interfere with or eliminate such properties or processes, and ideally enhances them.

Some ingredients may typically be processed in a way that leads to a particular particle size distribution. For example, caffeine commercially available in particulate form typically has a median particle size of roughly 15-20 microns (e.g., about 18 microns). Certain commercially-available vitamins in particulate form, such as B vitamins (e.g., B3 as niacin, B6 as pyridoxine, or B12 as cyanocobalamin), may also have a typical median particle size below a desirable range. This may also be the case for other ingredients, such as the sweeteners stevia and/or thaumatin. Any such “pre-existing” distributions may not be ideal for the aerosol product being prepared and its intended use. Compositions and methods have been discovered that can lead to substantially increased mean (or median) particle sizes of powders.

A consumable oil, when added to certain powdered ingredients, has been found to play a role in shifting the particle size distribution “upward” (i.e., to larger values), and to play a role in reducing the number/mass of smaller particles relative to larger particles. A hypothesis as to why this occurs is that smaller particles agglomerate onto each other, and/or into the oil, and/or onto larger particles, and/or “self-agglomerate.” creating relatively stable larger particles. Upon intake of such powders using an aerosol delivery device, it was found that often the powder was less likely to hit the back of the throat and thus less likely to elicit an unpleasant sensation or coughing.

In certain embodiments, agglomeration methods may use any one or more consumable oils, including, but not limited to, Aloe Vera, Artichoke Oil, Black Currant Seed Oil 14% GLA, Black Currant Seed Oil 15% GLA, Borage Oil 20% GLA, Borage Oil 22% GLA, Boswellia Serrata Oil, CLA Conjugated Linolic Acid 75% min., Evening Primrose Oil 10% GLA, Evening Primrose Oil 9% GLA, Flax Seed Oil 50% ALA, Garlic Oil, Grape Seed Oil, Guggul Lipid Oil, Olive Leak Extract, Oregano Oil, Perilla Oil 60% ALA, Pumpkin Seed Oil, Pygeum Oil, Rosehip Oil, Rosemary Oil, Saw Palmetto Oil, Sterols, Tocotrienol Palm Oil, Walnut Oil, Wheat Germ Oil, Sesame Seed Oil, Dill Seed Oil, Clove Bud Oil, Ginger Root Oil, Cinnamon Leaf Oil, Fennel Seed Oil, Curcuma Longa Oil, Cummin Seed Oil, Celery Seed Oil, Coriander Seed Oil, Red Raspberry Seed Oil, Cranberry Seed Oil, Blackberry Seed Oil, Cod Liver Oil (2500A/250D), Fish Oil 30% EPA/20% DHA, Fish Oil Concentrated, Fish Oil Deodorized, Marine Lipid Oil 18/12, Marine Lipid Oil 30/20, Marine Lipid Oil 36/24, Salmon Oil 18% EPA/12% DHA, Squalene Oil (Shark), Corn Oil, Vegetable Oil, Alpha Lipoic Acid, Cetyl Myristoleate CM, Coenzyme Q10, Lecithin, and/or Medium Chain Triglycerides MCT, naturally flavored Oils, and/or artificially flavored Oils. See, for example, U.S. Patent Application Ser. No. 61/643,871 and U.S. Patent Application Ser. No. 61/643,876, both incorporated herein in their entirety.

In certain embodiments, ingredients processed to a certain size may be further processed in combination with one or more additional ingredients. It is contemplated that a desired particle size, distribution and density can be acquired by, for example, agglomeration techniques as known to those in the art. The resultant agglomerated heterogenous particle can be combined into a specific formulation using other homogenous or heterogenous particles acquired by milling, spray drying, or agglomeration, and combined in a batch process. It is further contemplated that certain embodiments may use any one technique, or any combination of two or more particle production techniques, for the production of particles in the size distribution and population density desired. For example, agglomeration techniques can result in a subset of ingredients having the desired particle size, another agglomeration preparation can result in the remainder of ingredients having the desired particle size, and the two processed particles can be quantitatively combined in a batch process. Alternatively, agglomerated particles can be combined with another preparation of other ingredients (spray dried, for example) to develop a final consumable product formulation. Dry powder particles could be created from a single ingredient, such as chocolate, coffee, or truffles, or from a combination of ingredients. In the case of chocolate, chocolate bars, chocolate powder, cocoa powder, and other forms and varieties of foods derived from the cocoa plant may be used. In addition, in some cases, spices and other (natural or artificial) flavorings may be used alone or in combination with such food ingredients to create other tastes or sensations (e.g., natural or artificial chocolate, raspberry, mango, mint, vanilla, cinnamon, caramel, and/or coffee flavors). Depending on the food product(s) and device(s) used, the food product may be stored and/or contained in the form of a tablet or pill, in a blister pack, within a capsule, as simply a powder in a jar-like container, and/or in a tray, box, container, thermos, bottle, etc.

Formulations

It is desirable that formulations of the payload have a pleasant flavor, deliver effectively a desired target payload to the consumer, have stability both with respect to ingredient flavoring and biological activity, and have commercial stability with respect to manufacturing, processing and commerce activity (e.g., warehousing and transport). Ingredients are broadly considered as flavoring agents, dietary supplements, and target payloads including, but not limited to, energy products, over-the-counter pharmaceuticals, prescription pharmaceuticals, antioxidants, sleep-aids, weight-loss products, nutraceuticals, oral health compounds, and novelty products. While some of the compounds included herein are included and described under one category, it is understood that, based on the ingredient function, individual ingredients can be included in more than one category. For example, Vitamin C is considered both a vitamin and an anti-oxidant, or quercitin is considered an energy supplement as well as an anti-oxidant.

Embodiments of the present invention are drawn to formulations for the delivery of ingestable, aerosolizable powders with specific target payloads. Formulations can vary substantially to optimize payload delivery and performance, such as solubility, user experience (e.g., a desired flavoring, minimizing bitterness or unpleasant tastes or odors), and target payload activity (e.g. combinations of ingredients to enhance a particular effect, for example as a sleep aid, an energy compound, or kinetics of absorption of a pharmaceutical product). For example, a target payload may have a particular flavoring agent (for example a sweetener) that another target payload (for example, an ingredient already providing sweetened experience to the user) would not require for palatable flavor, or would require in different concentrations for a pleasant consumption experience, and/or for the desired delivery and performance characteristics. Flavoring agents, as used herein, can include, but are not limited to, a masking agent, an artificial sweetener, a natural sweetener, a flavor compound, an acidulant, and combinations thereof.

Generally, masking agents are used to manage or deflect taste, odor, visual characteristics of the payload, alter the mouth feel of the delivered payload, and to generate certain sensory perceptions of the payload.

Certain embodiments of the masking agents contemplated in the present invention include, but are not limited to, B-cyclodextrin, glycyrrizin, polymers (methylcellulose, polyvinylpyrrolidone, hydroxymethylcellulose, carboxymethylcellulose, ethylmethylcellulose) vanilla, etc.

Natural and artificial sweeteners are generally used to sweeten the payload sensory perception, deflect sensory aspects away from an undesired perception to a desired sensory perception. Sweeteners can also be used as flavor enhancers. Generally, for embodiments of the present invention, the choice of sweetener can be part of the overall characteristics desired in the aerosolizable powder (performance, user perception, manufacturing and commerce requirements, etc). Natural sweeteners are produced directly from natural products (plants), wherein artificial sweeteners are synthesized de novo or are modified natural sweeteners.

Certain embodiments of natural and artificial flavors contemplated in the present invention include, but are not limited to, stevia rebaudioside A, glycyrrizin, thaumatin, sorbitol, erythritol, mannitol, monk fruit, pentadin, xylitol, brazen, sugar, dextrose, crystalline fructose, maltodextrin, trehalose, molasses, aspartame, aspartame acesulfame salt, neotame, acesulfame, saccharin, sucralose, neohesperidin dihydrochalcone, sodium, saccharin, cyclamates, alitame and dulcim.

Flavoring compounds, as used herein, may be used to give the formulation payload a taste preferred by the end user, increase or enhance particular flavors or the perception of flavors. Flavors choices can include any fruit or vegetable flavor, or any artificial flavor, to elicit a desired taste perception (sweet, sour, bitter, salty and/or umami, and associated food or flavoring, e.g. mint, taste), as well as herbal or plant flavors that can otherwise be considered non-food (eg. Cinnamon), such as coffee, chocolate, and other confectionary flavors. Other flavor compounds considered as a novelty flavoring, including beer and other alcoholic beverages, hemp, vomitus, and novel combinations of flavors (e.g., beer flavoring with caffeine).

Acidulants, as used herein, may be considered as additives or compounds that change or maintain the final product acidity or alkalinity. Acidulants can be organic or mineral acids, bases, neutralizing agents or buffering agents.

Acidulants contemplated as embodiments in the present invention include, but are not limited to, citric acid, malic acid, lactic acid, glycolic acid, tartaric acid, fumaric acid, oxalacetic acid, succinic acid, lactoisocitric acid, shikmik acid, eulagic acid and/or glutamic acid. Another embodiment may be sodium bicarbonate, also serving the dual role of providing an effervescence effect to the palate and/or tongue upon contact dissolution.

Generally, dietary supplements may be considered as vitamins and/or minerals taken in addition to naturally obtained vitamins/minerals in food. Dietary supplements are taken 1) to enhance the physical well-being or state of health of the end user, 2) as a health related supplement, or 3) as supplements required to enhance deficient vitamin/mineral states in the end user. Dietary supplements can also add to a higher quality or perceived quality of the health state of the end user.

In certain embodiments, dietary supplements include, but are not limited to, Ascorbic Acid (Vitamin C), B Vitamins, Biotin, Fat Soluble Vitamins, Folic Acid, HCA (Hydroxycitric Acid), Inositol, pyruvate, Mineral Ascorbates, Mixed Tocopherols, Niacin (Vitamin B3), Orotic Acid, PABA (Para-Aminobenzoic Acid), Pantothenates, Pantothenic Acid (Vitamin B5), Pyridoxine Hydrochloride (Vitamin B6), Riboflavin (Vitamin B2), Synthetic Vitamins, Thiamine (Vitamin B1), Tocotrienols, Vitamin A, Vitamin D, Vitamin E, Vitamin F, Vitamin K, Vitamin Oils, Vitamin Premixes, Vitamin-Mineral Premixes, Water Soluble Vitamins, arsenic, boron, calcium, chloride, chromium, cobalt, copper, fluorine, iodine, iron, magnesium, manganese, molybdenum, nickel, phosphorous, potassium, selenium, silicon, sodium, strontium, sulfur, vanadium and zinc.

Target Payloads

Target payloads are contemplated for which an end user will obtain a desired effect upon ingestion of the specific powder compound. Target payloads broadly include, but are not limited to, energy supplements, over-the-counter pharmaceuticals, prescription pharmaceuticals, antioxidants, sleep-aids, weight-loss products, nutraceuticals, oral health compounds, and novelty product compounds. The payloads can be used in combination with other formulation compounds that are used in the manufacture of the edible cartridge. A combination of compounds in the edible cartridge and in the payload can allow for delivery of a full dosage of an OTC, for example, or full daily recommended allowance of a vitamin, mineral or supplement, that otherwise may not be efficient for delivery in aerosolized powder form in the cartridge as described herein.

Energy supplements for use as described herein are designed to boost mental or physical activity. Various embodiments of ingestible powdered formulations for the present invention include, but are not limited to, American ginseng, Red ginseng, Siberian ginseng, maca, rhodiola, ginger, guarana, turmeric, acetyl-L-carnitine, L-carnitine, creatine, taurine, L-phenylalanine, L-arginine, tyrosine, acetyl-tyrosine, N-acetyl L-tyrosine, ginko biloba, yerba-mate, kola nut, gotu kola, maitake, cordyceps sinensis, guarana, acai-berry, L-theanine, caffeine, quercitine, synephrine, green tea extract, theophylline, epigallocatechin gallate (EGCG), capsaicin, bee pollen, alpha-lipoic acid, and 1,3 dimethylamylamine (geranium), D-ribose, Fo-Ti, cha de bugre extract and St. Johns wort.

Oral health compounds contribute to decreasing unwanted bacterial flora and/or covering up unwanted odors and/or flavors. Control of the unwanted flora decreases incidence of tooth decay, halitosis, and potentially contributes to long-term health benefits including incidence of heart disease.

In certain embodiments, oral health compounds for use in the present invention include, but are not limited to, fluoride, vitamin C, vitamin B, zinc, menthol, thymol, eucaleptic, sodium bicarbonate, vitamin K, chlorhexidine, and xylitol.

Weight loss compounds are commonly divided into groups categorized as appetite suppressants, acting to manipulate hormonal and chemical processes in the body that otherwise increase hunger and/or the sense of feeling satiated (e.g. anorectics such as epinephrine and norepinephrine/noradrenaline), fat or cholesterol uptake inhibitors (such as green tea extract), gastrointestinal fillers, and thermogeneic compounds which boost a normal metabolic rate of the individual and result in metabolism of fat stores, all of which are contemplated for use in the present invention. Weight loss compounds can be synthetic or natural.

In certain embodiments of the present invention, weight loss compositions contemplated herein include, but are not limited to, hoodia, chitosan, chromium picolinate, conjugated linoleic acid, glucomannan, green tea extract, guar gum, guarana, guggal, senna, ephedra, bitter orange, fucoxanthin, white bean extract, vitamin D, human chorionic gonadotropin, resveratrol, capsaicin, chia, hoodia, L-carnitine, raspberry ketones, banaba leaf, red clover, ginger, almonds, acai berry, flax seeds, leucine and lipodrene.

Sleep-aid compounds assist in slowing the metabolic resting rate of an individual to allow one to relax and gain more restful or longer sleep periods. In certain embodiments of the present invention, sleep aid compositions contemplated herein include, but are not limited to melatonin, 5-hydroxytryptophan, 5-hydroxytrypatmine, diphenhydramine, doxylamine, benzodiazepine, kava, serenite, chamomile, phenibut, catnip herb, chamomile, glycine, hops, L-theanine, L-tryptophan, glycine, GABA and valerian.

Various over the counter and prescription based (pharmaceutical) drugs are contemplated for easier ingestion, and in some instances a more pleasant taste is experienced by the user.

Additionally, because of the dispersion of the powder upon aersolization, increased kinetic and/or metabolic uptake may be experienced by the user owning to the greater surface area of the dosed compound. In many instances, for example during an allergic reaction, increased kinetics of drug activity may be desirable.

In certain embodiments, over-the-counter (OTC) and prescription (pharmaceutical) drugs include, but are not limited to, amikacin, gentamicin, kanamycin, neomycin, netilmicin, tobramycin, paromomycin, geldanamycin, herbimycin, loracarbef, ertapenem, doripenim, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, telavancin, clindamycin, lincomycin, daptomycin, azithromycin, clarithromycin, dirithromycin, erythromycin, roxithromycin, troleandomycin, telithromycin, spectinomycin, aztreonam, furazolidone, nitrofurantoin, amoxicillin, ampicillin, azlocillin, carbenicillin, cloxacillin, dicloxacillin, flucloxacillin, mezlocillin, methicillin, nafcillin, oxacillin, penicillin, piperacillin, temocillin, ticarcillin, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacine, temafloxacin, mafenide, sulfonamidochrysoiodine, sulfacetamide, sulfadiazine, silver, sulfadiazine, sulfamethizole, sulfamethoxazole, sulfanilamide, sulfasalazine, sulfisoxazole, trimethoprim, trimethoprim-sulfamethoxazole, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, linezolid, metronidazole, mupriocin, platensimycin, quinupristin/dalfopristin, rifaximin, thiamphenicol, tigecycline, tinidazole, Fluoxetine, sertraline, paroxetine, fluvoxamine, citalopram, escitalopram, mirtazapine, triazolam, quazepam, estazolam, temazepam, zolpidem eszopiclone zalepon, Trazodone, Citalopram, escitalopram, desvenlafaxine, duloxetine, milnacipran, venlafaxine, tramadol, sibutramine, etoperidone, lubazodone, nefazodone, trazodone, reboxetine, viloxazine, atomoxetine, bupropion, dexmethylphenidate, methylphenidate, amphetamine, dextroamphetamine, dextromethamphetamine, lisdexamfetamine, amitriptyline, butriptyline, clomipramine, desipramine, dosulepin, doxepin, imipramine, iprindole, lofepramine, melitracen, nortriptyline, opipramol, protriptyline, trimipramine, amoxapine, maprotiline, mianserin, mirtazapine, isocarboxazid, moclobemide, phenelzine, selegiline, tranylcypromine, pirlindone, busipirone, tandospirone, aripiprazole, vilazodone, quetiapine, agomelatine, nefazodone, quetiapine, asenapine, carbamazepine, lithium, olanzapine, valproic acid, alprazolam, lorazipam, chlordiazepoxide, clonazepam, etizolam, tofizopam, Azelastine, cetirizine, clemastine, desloratadine, dimenhydrinate, diphenhydramine, doxylamine, fexofenadine, loratadine (Claritin), ketorolac tromethamine, pemirolast potassium, ketotifen, neodocromil sodium, loteprednol etabonate, ipratropium bromide, beclomethasone, dexamethasone, epinastine, fluticasone, oxymetazoline, triamcinolone, cromolyn sodium, flunisolide, mometasone, ciclesonide, carbinoxamine maleate, olopatadine, budesonide, montelukast, clemastine, epinephrine, fluticasone furoate and levocetirizine, Celecoxib (Celebrex), etodolac (iodine), meloxicam (Mobic), rofecoxib (Vioxx), valdecoxib (Bextra), ibuprofen, naproxen, diclofenac, flurbiprofin, indomethacin, ketoprofen, ketorolac, nabumetone, oxaprozin, piroxicam, sulindac, Aspirin, Acetaminophen, Pseudoephedrine HCl, Dextromethorphan, Chlorpheniramine Maleate, Pseudoephedrine HCl, Xylometazoline, Benzododecinium, Butamirate citrate, Clemastine, diphenynhydramine citrate, diphenynhydramine, Chiorpheniramine Maleate, Dextromethorphan Hydrobromide, Oxymetazoline hydrochloride, guaifenesin, ibuprofin, phenylephrin, Acid production control (omeprazole), laxative (loperimide) smoking (nicotine), Ezetimibe, Simvastatin, Eptifibatide, Sitagliptin, Metformin, Losartan Potassium, Hydrochlorothiazide, Finasteride, Enalapril maleate, Hydrochlorothiazide, raltegravir, peginterferon alpha-2b, caspofungin acetate, imipenem and cilastatin sodium, ertapenem sodium, moxifloxacin, posaconazole, Indinavir sulfate, efavirenz, ribavirin USP, peginterferon alfa and ribavirin, rizatriptan benzoate, dorzolamide hydrochloride, Montelukast sodium, infliximab, mometasone furoate monohydrate, desloratadine, etoricoxib, mometasone furoate, golimumab, albuterol sulfate, mometasone furoate/formoterol fumarate, temozolomide, fosaprepitant dimeglumine, Interferon alfa-2b, Gardasil™, ProQuad™, MMR II™, Varivax™, RotaTeq™, Pneumovax™, Zostavax™, alendronate sodium, etonogestrel/ethinyl estradiol, follitropin beta, etonogestrel, desogestrel, Zelephon, Zolpidem Tartrate, estazolam, flurazepam, temazepam, eszopiclone, zaleplon, zolpidem, Ramelteon, amitriptyline, doxepin, mirtazipine and trazodone. Various other compounds are contemplated for use as target payload ingredients in ingestable powder formulations. For example, antioxidants, hormones and other proteins, enzymes, amino acids, probiotics, etc., are desirable target payloads.

In certain embodiments, hormones are used for hormone replacement and supplementation. Various hormones contemplated for use in the invention described herein include, but are not limited to, apidonectin, aldosterone, androgen, natriuretic peptide, 7-Keto-DHEA, Androstenedione, dihydroepiandrosterone (DHEA), Melatonin, Nor-Androstenedione, pregnenolone, progesterone, 19 Nor-4-Androstendiol, 19 Nor-4-Androstenedione, 19 Nor-5-Androstenediol, 19 Nor-5-Androstendione, 3-Indolebutyric Acid, 4 Androstendiol, 4 Androstendione, 6 Furfurylaminopurene, 6-Benzylaminopurine, calcitonin, cortisol, erythropoietin, gonadotropin, human growth hormone (HGH), incretins, leptin, lutenizing hormone, orexin, parathyroid hormone, pregnenolone, progesterone, prolactin, relaxin, renin, testosterone, and vasopressin.

In certain embodiments, enzymes and amino acids are contemplated, and include, but are not limited to, alpha galactosidase, amylase, bromelain, cellulase, papain, peptidase, protease, proteolytic enzymes, superoxide dismutase, trypsin, betaine, casein, glutamic Acid, L-alanine, L-arginine, L-cysteine, L-glutamine, L-glycine, L-histidine, L-isoleucine, L-leucine, L-lysine, L-methionine, L-ornithine, L-phenylalanine, L-proline, L-taurine, L-threonine, L-tryptophan, L-tyrosine, L-valine, N-acetly-L-cysteine, protein soluble soy, soy protein isolates, and whey protein isolates.

In certain embodiments, antioxidants for use in powder formulations include, but are not limited to, carotenoids, flavonoids, isoflavones, tocopherol, tocotrienol, lipoic acid, melatonin, superoxide dismutase, coenzyme Q10, alpha lipoic acid, vitamin A, chromium biotin, selenium and ascorbic acid.

In certain embodiments, carotenoids contemplated for use in the present invention include alpha-carotene, beta-carotene, cryptoxanthin, lycopene, lutein, zeaxathin, apocarotenal astaxanthin, canthaxanthin, lutein/lutein esters, etc.

In certain embodiments of the present invention, flavonoid used in the formulations include esveratrol, quercetin, rutin, catechin, proanthocyanidins, acai berry extract, raspberry extract, cranberry extract, pomegranate extract, plum extract, cherry extract, rosemary extract, etc.

In certain embodiments, isoflavones are used, including, but not limited to, genistein, daidzein, biochanin A, and formononetin.

Further embodiments for formulations of dry powders include probiotics to re-establish healthy intestinal bacterial flora. In certain embodiments, probiotics for use in the present invention include, but are not limited to, Bacillus coagulans GBI-30, 6086, Bifidobacterium animalis subsp. lactis BB-12, Bifidobacterium longum subsp. inantis 35624, Lactobacillus acidophilus NCFM, Lactobacillus paracasei Stl 1 (or NCC2461), Lactobacillus johnsonii NCC533), Lactobacillus plantarum 299v, Lactobacillus reuteri ATCC 55730 (Lactobacillus reuteri SD2112), Lactobacillus reuteri Protectis (DSM 17938, daughter strain of ATCC 55730), Saccharomyces boulardii, Lactobacillus rhamnosus GR-1 & Lactobacillus reuteri RC-14, Lactobacillus acidophilus NCFM & Bifidobacterium bifidum BB-12, Lactobacillus acidophilus CL1285 & Lactobacillus casei LBC80R, Lactobacillus plantarum HEAL 9 & Lactobacillus paracasei 8700:2, Lactobacillus bulgaricus, Streptococcus thermophiles, and/or Bifidobacterium spp. Plants and plant extracts can provide compositions for dietary supplements, energy products, antioxidants, sleep-aids, weight-loss products, nutraceuticals, oral health compounds, novelty products, etc. Such compositions may be categorized as botanical supplements and botanical extracts. Aqueous or oil based botanical supplements can be combined at low volume with powdered components and or be used in, for example, agglomeration processes.

In certain embodiments, botanical extracts and plant-based supplements include, but are not limited to, Acerola Extracts, Alfalfa, Blue Green algea, Aloe, Amla, Angelica Root, Bacopa Monnieri, Mucuna Pruriens, Anise Seed, Arnica, Artichoke, Ashwagandha, Astragalus, Ayurvedic Herbs, Barberry, Barley Grass, Barley Sprout Extract, Benzoin, Bilberry, Bioflavonoids, Bitter Melon, Bitter Orange, Black Cohosh, Black Currant, Black Walnut, Bladderwrack, Blue Cohosh, Blueberry, Boswellia, Brahmi, Broccoli, Burdock, Butcher's Broom, Calendula, Capsicum, Cascara Sagrada, Cat's Claw, Catnip herb, Cayenne, Celery Seed, Certified Organic Herbs, Chamomile, Chapparal, Chaste Berry, Chicory Root, Chinese Herbs, Chlorella, Chlorophyll, Citrus Aurantium, Cocoa, Coriander, Corn Silk, Cranberry, Curcuminoids, Damiana, Dandelion, Devil's Claw, Diosgenin, Dong Quai, Echinacea, Elderberry, Elecampane Root, Ephedra, Essential Oils, Eucalyptus, Evening Primrose, Eyebright, Fennel, Fenugreek, Feverfew, Flax Products, Garcinia, Cambogia, Garlic, Gentian, Ginger, Ginkgo, Biloba, Ginseng (American), Ginseng (Panax), Ginseng (Siberian), Goldenseal, Gotu Kola, Grape Seed Extract, Grape Skin Extract, Grapefruit Seed Extract, Green Food Products, Green Lipped Mussel Powder, Green Tea, Griffonia simplicifolia, Guarana, Guggul, Gymnema Sylvestre, Hawthorne, Herbal Extracts, Herbal Teas, Hops, Horehound, Horse Chestnut, Horsetail, Hysop, Ipriflavone, Jojoba Oil, Juniper Berries, Kava Kava, Kelp Extract, Kombucha, Kudzu, Larch, Lavender, Lemon Balm, Licorice Extract, Linden Flowers, Lobelia, Maca, Maitake Mushroom, Marshmallow, Milk Thistle, Molasses, Mushrooms, Neem, Nettle, Noni, Nopal, Oatstraw, Octacosanol, Olive Extract, Orange Peel Extract, Oregano Oil, Oregon Mountain Grape, Organic Sweeteners, Parsley, Passion Flower, Pau d'Arco, Pennyroyal, Peppermint, Pfaffia Paniculata, Pine Bark Extract, Piper Longum, Pygeum Africanum, Quercitin, Raspberry Powder, Reishi Mushroom, Resveratrol Extract, Rhubarb Root, Rice Products, Rose Hips, Rosemary Extract, Sage, Sarsaparilla, Saw Palmetto, Schizandra, Seaweed extracts, Senna, Shatavari, Shiitake Mushroom, Silymarin, Skullcap, Slippery Elm, Soy Isoflavones, Soybean Products, Spirulina, St. John's Wort, Stevia, Summa, Tea Tree Oil, Terminalia ajruna, Tribulus terrestris, Triphala, Tumeric, Uva Ursi, Valerian Extract, Vegetable Extracts, Vitex, Wheat Germ, White Willow Bark, Wild Cherry bark, Wild Yam, Witch Hazel, Wormwood, Yarrow, Yellow Dock, Yerba Sante, Yohimbine, Yucca, 20-ECD 7-9%, Acetyl L-Camitine HCl 99%, 4-Androstenedione 99%, Adenophora Tetraohylla Ext 5:1, Alisma Extract 10:1, Alpha Lipoic Acid 99%, Angelica Root Extract, Arbutin 99%, Artemisia Extract 4:1, Artichoke Extract 5%, Globe Asparagus Extract 4:1, Asparagus Powder, Astragulus Extract 10:1, Astragulus Extract 4:1, Astragulus Extract 5:1, Astragulus Root Extract 0.5%, Astragulus Root Powder, Atractylodes Extract 10:1, Avena Sativa Extract 10:1, Avena Sativa Extract 4:1, Barbed Skullcap Extract 10:1, Barberry Extract 10%, Bee Pollen Powder, Beta-Sisterol 35%, Bilberry Extract 10:1, Bitter Melon Extract 8:1, Black Cohosh Extract 2.5%, Black Cohosh Root Powder, Black Pepper Extract 4:1, Black Soy Bean Extract 10:1, Bone Powder, Boswellia Serrata Extract 65%, Broccoli Sprout Extract 10:1, Buchu Leaf Powder, Buplerum (Chai Hu) Extract 5:1, Burdock Root Extract 4:1, Cabbage Extract 4:1, Caffeine (Natural) 86-87%, Caffeine 99%, Calcium Citrate Granular 21%, Calcium-Pyruvate 99%, Carrot Root Extract 4:1, Cassia Nomame Extract 4:1, Catnip Extract 4:1, Cat's Claw (Inner Bark), Powder Cauliflower Extract 4:1, Celandine (Greater) Extract 4:1, Celery Seed Extract, Cetyl Myristoleate 11%, Cetyl Myristoleate 20%, Chaenomeles Extract 4:1, Chamomile Flower Extract 10:1, Chamomile Flower Extract 4:1, Chaste Tree Berry Extract 4:1, Chitin Chitosan 80%, Chitosan 90%, Chondroitin Sulfate 90%, Chrysin 99%, Cinnamon Powder, Cistanches Extract 5:1, Citrus Aurantium Extract 6%, Citrus Bioflavonoid Complex 13%, Citrus Peel Extract 5:1, Clove Extract 5:1, Clove Powder, Coca Extract 4:1, Codonopsis Pilosula Extract 5:1, Colostrum, Common Peony Extract 8:1, Cordyceps Extract 7%, Cornsilk Extract 4:1, Cornsilk Powder, Corydalis Extract 10:1, Cranberry Extract 4:1, Cranberry Powder, Curcumin Extract 95%, Cuscuta Extract 5:1, Damiana Extract 4:1, Damiana Leaves Powder, Dandelion Powder, Dandelion Root Extract 6:1, Danshen Extract 80%, D-Calcium Pantothenate, Devil's Claw Extract 2.5%, Devil's Claw Extract 4:1, Devil's Claw Root Powder, DHEA 99%, Diosgenin 95%, DL-Phenyl Alanine, DMAE Bitartrate, Dong Quai Extract 10:1, Dong Quai Extract 4:1, Dong Quai Root Powder, D-Ribose, Echinacea Angustifolia Extract 4:1, Echinacea Leaf Powder, 10 Echinacea Purpurea Extract 10:1, Echinacea Purpurea Extract 4%, Echinacea Purpurea Extract 4:1, Echinacea Purpurea Root Powder, Elder Flower Extract 4:1, Elderberry Extract 20:1, Elderberry Extract 4:1, Epimedium Extract 10%, Epimedium Extract 10:1, Epimedium Extract 4:1, Epimedium Extract 5%, Epimedium Powder, Eucommia (Du Zhong) Extract 5:1, Fennel Seed Extract 4:1, Fennel Seed Powder, Fenugreek Extract 4:1, Fenugreek Extract 6:1, Feverfew Extract 5:1, Fisetin, Fish Oil Powder, Forbidden Palace Flower Extract 5:1, Forskolin 8%, Fo-Ti Extract 12:1, Fo-Ti Extract 8:1, Fo-Ti Powder, Gardenia Extract 8:1, Garlic Extract 4:1, Garlic Powder, Gentian Root Extract 6:1, Ginger Extract 4:1, Ginger Root Extract 5%, Ginger Root Powder, Ginkgo Biloba Extract 8:1, Ginkgo Extract 24/6%, Ginkgo Extract 24/6% <5, Ginkgo Extract 24/7%, Ginkgo Leaf Extract 4:1, Ginkgo Leaf Powder, Ginseng (Korean) Powder, Ginseng (Panax) Extract 5%, Ginseng (Panax) Extract 8%, Ginseng (Panax) Extract 80%, Glucomannans Konjac Powder, Glucosamine HCl 95%, Granulation Glucosamine HCl 99%, Glucsosamine Sulfate Potassium, Glucsosamine Sulfate Sodium 95%, Granulation Glucsosamine Sulfate Sodium 99%, Goldenrod Extract 4:1, Goldenrod Powder, Goldenseal Root Extract 14%, Goldenseal Root Powder, Gotu Kola Extract 16%, Gotu Kola Extract 4:1, Gotu Kola Extract 8:1, Gotu Kola Powder, Grape Fruit Powder, Grape Seed, Grape Seed Extract 10:1, Grape Seed Extract 20:1, Grape Seed Extract 4:1, Grape Seed Extract 5:1, Grape Seed Extract 95%, Grape Seed Powder, Grape Skin Extract 20:1, Grape Skin Extract 4:1, Grass-Leaved Sweetflai Extract, Green Lip Mussel Extract, Green Tea Extract 30%, Green Tea Extract 4:1, Green Tea Extract 95%, Guarana Seed Extract 10%, Guarana Seed Extract 22%, Guarana Seed Extract 25%, Guggul Extract 10%, Guggul Extract 2.5%, Gugulipid Extract 10%, Gymnema Sylvestre Extract 25%, Gymnema Sylvestre Powder, Hawthorne Berry Extract 4:1, Hawthorne Berry Powder, Hawthorne Leaf Extract 2%, Hearbacious Peony Extract 5:1, Hesperidin Extract 98%, Honeysuckle Herb Extract 4:1, Hops Flower Extract 4:1, Horehound Extract 10:1, Horehound Extract 4:1, Horehound Herb Powder, Horse Chestnut Extract 20%, Horse Chestnut Extract 4:1, Horse Chestnut Powder, Horsetail Extract 7%, Horsetail Powder, Houttuynia Cordata Extract 5:1, Hydrangea Extract 8:1, Hydroxy Apatite, Hyssop Extract 4:1, Indole-3-Carbinol 99%, Isodon Glaucocalyx Extract 10:1, Japanese Knotweed Extract, Jiaogulan Extract 4:1, Jin Qian Cao Extract 4:1, Jingjie Extract 4:1, Jujube Fruits Extract 4:1, Kava Kava Extract 30%, Kava Kava Powder, Kelp Extract 4:1, Kelp Powder, Kidney Bean Extract 10:1, Kidney Bean Pole 4:1, Kidney Bean Pole 8:1, Kidney Bean Powder, Kola Nut Extract 10%, Kudzu Extract 4:1, Kudzu Extract 6:1, Lettuce Extract 4:1, L-Glutamine, L-Glycine, Licorice Extract 10%, Licorice Extract 5:1, Licorice Powder, Lotus Leaf Powder, L-Tyrosine, Lycium Fruit Extract 4:1, Lycium Fruit Extract 5:1, Ma Huang Extract 6%, Ma Huang Extract 8%, Maca Extract 0.6%, Maca Root Powder, Magnesium Stearate, Magnolia Bark Powder, Magnolia Officinal Extract 4:1, Maca Extract 4:1, Maitake Mushroom Extract 4:1, Marigold Extract (Lutein 5%), Methozyisoflavone 99%, Methylsufonylmethane 99%, Milk Thistle Extract 4:1, Milk Thistle Seed Extract 80% silymarin, Morinda Extract 5:1, Motherwort Extract 4:1, Motherwort Powder, Mucuna Pruriens Extract (15% L-Dopa), Muira Puama Extract 12:1, Muira Puama Extract 4:1, Muira Puama Powder, Mushroom Extract 10:1 (feishi), Mustard Seed Extract 8:1, Myrobalan Extract 4:1, Myrrha Gum Extract 2.5%, N-Acetyl-D-Glucosamine, N-Acetyl-L-Cysteine, Nettle Extract 7%, Nettle Leaf Extract 4:1, Nettle Leaf Powder, Noni Powder, Olive Leaf Extract 18%, Olive Powder Orange Peel Extract 4:1, Orange Peel Powder, Oroxylum Indicum Extract 4:1, Oroxylum Indicum Powder, Oyster Meat Powder, Oyster Shell Powder, Papaya Fruit Extract 4:1, Parsley Extract 10:1, Parsley Extract 4:1, Parsley Leaf Extract 4:1, Parsley Powder, Passion Flower Extract 4:1, Passion Flower Powder, Pau D'Arco Powder, Peppermint Extract 4:1, Peppermint Powder, Perilla Seed Extract 4:1, Periwinkle Extract 4:1, Pharbitidis Extract 4:1, Phosphatidyl Serine 20%, Pine Bark Extract 4:1, Plantago Asiatica Leaf Extract 5:1, Polygala Tenoifolia Extract 4:1, Polygonum Extract, Polygonum Extract 4:1, Pregnenolone 99%, Propolis Extract 3%, Pseudoginseng Extract, Psyllium extract 4:1, Pumpkin Seed Extract 4:1, Purple Willow Bark Extract 4:1, Purslane Herb Extract 4:1, Pygeum Extract 4:1, Quercetin, Radish Extract 4:1, Radix Isatidis Extract 4:1, Radix Polygoni Extract 4:1, Red Clover Extract 4:1, Red Pepper Extract 4:1, Red Yeast Rice, Red Yeast Rice Extract 10:1, Red Yeast Rice Powder, Rehmannia Root Extract 4:1, Reishi Mushroom Extract 4:1, Rhodiola Rosea Extract 4:1, Rhododendron Extract 4:1, Rhododendron Powder, Rhubarb Extract 4:1, Rhubarb Root Powder, Riboflavin (B2), Rice Powder, Rosemary Extract 20%, Rumex Madaid Extract 4:1, Salvia Extract 10:1, Salvia Extract 4:1, SAMe, Saw Palmetto Extract 25%, Saw Palmetto Extract 4:1, Saw Palmetto Extract 45-50%, Saw Palmetto Oil 85-95%, Saw Palmetto Powder, Schizandra Extract 10:1, Schizandra Extract 4:1, Scopolia Acutangula Powder, Sea Cucumber Powder, Senna Leaf Powder, Sesame (Black) Seed Powder, Shark Cartilage Powder, Shitake Mushroom Extract, Siberian Ginseng Extract 0.8%, Siberian Ginseng Extract 4:1, Siberian Ginseng Powder, Skullcap Extract 4:1, Skullcap Extract 4:1, Slippery Elm Powder, Sodium-Pyruvate 99%, Songaria Cynomorium Extract 4:1, Songaricum Powder, Spirulina Powder, St. John's Wort Extract 0.3%, St. John's Wort Extract 4:1, St. John's Wort Powder, Stanol 50%, Stephania Extract 4:1, Stevia Extract 4:1, Sulfate N+ Suma Root Extract 4:1, Suma Root Powder, Taurine Powder, Thorowax Extract 4:1, Tomato Extract, Tomato Extract (0.2% Lycopene), (trans)-Resveratrol 20-25%, Tribulus Extract 10:1, Tribulus Extract 40%, Tribulus Powder, Trifal Extract 4:1, Turmeric Extract 4:1, Turmeric Root Powder, Uva Ursi Extract 4:1, Uva Ursi Powder, Valerian Root Extract 0.8%, Valerian Root Extract 4:1, Valerian Root Powder, Vinca Major Seed Extract 10:1, White Wax Extract 4:1, White Willow Bark 15% (total salicins), White Willow Bark 20%, White Willow Bark 25%, White Willow Bark Extract 4:1, White Willow Bark Powder, Wild Yam Extract 10:1, Wild Yam Extract 16%, Wild Yam Extract 4:1, Wild Yam Extract 6%, Wild Yam Powder, Williams Elder Extract 4:1, Wolfberry Fruit Extract 10:1, Wolfiporia Extract 8:1, Yellow Dock Root Extract 4:1, Yerba Mate Extract (2% caffeine), Yerba Mate Extract 4:1, Yohimbe Bark Extract 15:1, Yohimbe Bark Extract 2%, Yohimbe Bark Extract 3%, Yohimbe Bark Powder, and Yucca Extract 4:1, Nutraceuticals are generally thought of as food or food product that reportedly provides health and medical benefits, including the prevention and treatment of disease, and can be defined as a product isolated or purified from foods that is generally sold in medicinal forms not usually associated with food. A nutraceutical may have a physiological benefit or provide protection against chronic disease. Such products may range from isolated nutrients, dietary supplements and specific diets to genetically engineered foods, herbal products, and processed foods such as cereals, soups, and beverages. With recent developments in cellular-level nutraceutical agents, researchers, and medical practitioners are developing templates for integrating and assessing information from clinical studies on complementary and alternative therapies into responsible medical practice.

In certain embodiments, nutraceuticals are used, including, but not limited to, 5-Hydroxytryptophan, Acetyl L-Carnitine, Alpha Lipoic Acid, Alpha-Ketoglutarates, Bee Products, Betaine Hydrochloride, Bovine Cartilage, Caffeine, Cetyl Myristoleate, Charcoal, Chitosan, Choline, Chondroitin Sulfate, Coenzyme Q10, Collagen, Colostrum, Creatine, Cyanocobalamin (Vitamin B12), DMAE, Fumaric Acid, Germanium Sesquioxide, Glandular Products, Glucosamine HCL, Glucosamine Sulfate, HMB (Hydroxyl Methyl Butyrate), Immunoglobulin (Immune System Support), Lactic Acid, L-Carnitine, Liver Products, Malic Acid, Maltose-anhydrous, Mannose (d-mannose), MSM, Other Carnitine Products, Phytosterols, Picolinic Acid, Pyruvate, Red Yeast Extract, S-adenylmethionine (SAMe), Selenium Yeast, Shark Cartilage, Theobromine, Vanadyl Sulfate, Velvet Deer Antler, Yeast, ATP, Forskolin, Sterol Esters, Stanol Esters, Probiotics, Lactoferin, Lutein Esters, Zeaxanthin, Immunoglobulins, Ipriflavone, Isoflavones, Fructo-Oligo-Saccharides, Inulin, Huperzine A, Melatonin, Medicinal Mushrooms, Bile Products, Peptone Products, Glandular Products, Pancreatic Products, Thyroid Products, Ribose, Probiotics, oleo resins, Dill Seed oleo resin, Black Pepper oleo resin, and Capsicum oleoresin,

In other powder embodiments, formulations may contain a processing aid, for example talc or Nu-FLOW® (Ribus, Inc. of St. Louis, Mo.) a synthetic aid with characteristics similar to silicon to allow for a desired flowability upon manufacturing and/or delivery during aerosolizing device actuation. Other excipients may include penetration enhancers and/or solubility agents including, but not limited to, tweens (polysorbates), spans (sorbitan esters), sodium lauryl sulfate and other surfactants.

For all formulations, component ingredients may serve to provide several functions. For example, certain botanical extracts and botanical products may have multiple functions (for example as a flavoring agent, dietary supplement, sleep aid and as an appetite suppressant). It is further understood that synergistic interactions between and among ingredients in the formulations may provide advantages in taste, product performance, manufacturing and handling attributes. Generally, formulations consider that taste, in addition to intended use or benefits of the product (providing energy/high caffeine, providing vitamins, providing flavor, etc.), as well as regulatory restrictions on the use of particular products in particular jurisdictions, all may contribute to the individual ingredient quantities and/or concentrations used.

Particle engineering can also account for reducing any tendency to cough, gag, or otherwise react unfavorably to the aerosol. For example, some payload formulations may result in a mouth feel/oral sensation that is more pleasant with a range of particle sizes greater than about 50 microns but less than 100 microns. Therefore, in particular embodiments, the range of particle sizes is engineered to the specific particle payload formulation to optimize aerosolizablity, solubility, and membrane transport.

Additionally, particle payload median size distribution can be engineered for good performance in a particular aerosolizing device. For example, in certain embodiments a particle size distribution of a particular formulation for use in a disposable device may be numerically larger or smaller than the same formulation for use in a disposable cartridge and/or than for use in an edible cartridge or device. In other embodiments, the particle size distribution is chosen for required, maximal and/or consistent payload delivery per actuation of a particular device. For example, a single device or cartridge may be designed for carrying any class of formulations (i.e., a pharmaceutical, an OTC, an energy supplement, etc.), but the particle size distribution and formulation of different payloads results in different payload delivery amounts per actuation of the same device.

These dry powder particles could be created from a single product or ingredient of a composition, such as chocolate, coffee, or truffles, or from a combination of foods or ingredients, such as combinations representative of an entire dish or meal (e.g., mixed fruits, pizza, pastry, meat and potatoes, etc.). In the case of chocolate, chocolate bars, chocolate powder, cocoa powder, and other forms and varieties of foods derived from the cocoa plant may be used. In addition, in some cases, spices and other (natural or artificial) flavorings may be used alone or in combination with such food ingredients to create other tastes or sensations (e.g., natural or artificial chocolate, raspberry, mango, mint, vanilla, cinnamon, caramel, and/or coffee flavors). Additionally, the device may contain a single dose of food product or multiple doses/portions of the food product. In addition, they may be made from largely liquid products, for example by extracting dissolved solids or using other solid components. In some embodiments, flavors can be experienced while using less of the actual product compared to normal ingestion. In addition, by mixing different powders, new flavors can be created.

In some embodiments, it is possible to deliver odors using appropriately designed and appropriately sized particles, which may be utilized independently or in addition to embodiments described herein, i.e., in addition to delivery of aerosolized food product so as to enhance the aesthetic experience.

“Food product”, “aerosol”, “particle”, and other similar terms are used throughout this document, and though they may typically refer to small solid particles derived from foods, these terms may in some cases refer to any of the other edible products including, but not limited to, a food product, an energy supplement, a pharmaceutical compound, an over-the-counter pharmaceutical compound, a nutraceutical, a sleep-aid compound, a weight-loss compound, an oral health compound, etc.

### Applications

Our device can transform how ingestible aerosolizable payloads are experienced, allowing for an enhanced delivery and performance of ingestible products. For example, medicines can be delivered more effectively, and by having larger surface area to volume in the aerosolized payload, are likely to be transported through membrane barriers at higher kinetic efficiencies. Additionally, for food-based payloads, the aerosolizing device can allow subjects to experience food either individually with hand held devices, or expose themselves to, for example, rooms filled with aerosolized clouds of various flavorings. The devices described herein can be applicable for compounds used for weight loss, OTC and pharmaceutical compound delivery (for example, allergy medicines, cold and flu medicines, etc.), nutraceuticals, dietary supplements, energy supplements, etc.

In some embodiments, the devices and methods described can allow subjects to experience food by exposing themselves to aerosolized food via individual, hand-held, and/or portable devices. In some embodiments, our technology may be used in and/or associated with social contexts similar to candy eating or cigarette smoking. For example, some embodiments may be carried about and used at various points throughout the day, or used simultaneously by multiple users.

In addition, the device can serve to provide nutrition to subjects either who are incapable of chewing or for whom delivery of food is not convenient. For example, the particle delivery device may be useful for elderly or young children, for whom chewing or feeding is inconvenient. In addition, individuals with medical conditions that require them to be fed in particular ways (e.g., by a feeding tube or intravenously) may use certain embodiments of this invention as a way to experience and taste food again.

In certain embodiments, the aerosolizing device can serve to facilitate the intake of medication that may not be of a pleasurable taste. If used in conjunction with delivery of the medication, e.g. orally, the device can provide an additional flavor that masks the flavor of the medication.

In some embodiments, the aerosolizing device described herein may be used for weight control or addiction mitigation applications. For example, the aerosolizing device can allow for subjects to consume relatively small or negligible quantities of food products or certain unhealthy or addictive substances, and the exposure to the particles (e.g., food particles, weight loss, nutraceuticals, etc.) via the device may provide a sensation or satisfaction normally associated with the consumption of a larger quantity of the food or substance in question, thereby potentially satisfying hunger or addictive urges without the (potentially negative) consequences of actually consuming larger amounts of the substance(s). In some cases, this may be due to the higher surface area of the food product exposed to surfaces of the mouth, for example exposed to taste receptors, relative to the overall quantity (e.g., mass) of food product. Indeed, the particle delivery device may form a basis for dieting, weight control and healthy eating programs (for example, by satisfying cravings for sweets, fatty foods, chocolate and caffeine) and addiction treatment (for example, by satisfying urges for alcohol, smoking, drugs but in much smaller, less harmful amounts).

Additionally, the particle delivery device can serve as a means for taste-testing a number of items in a simple and efficient way. For example, a patron at a restaurant can taste test various dishes on the menu before making a selection. Additionally, chefs may use the particle delivery device to test combinations of foods while cooking or designing a recipe. Similarly, the device may serve as an aid in cooking lessons, as an international “dining” experience for a subject, as a way to teach children about food, etc. In addition, the particle delivery device may be used to improve quality of life, for example, with respect to individuals subject to special dietary restrictions

Other useful applications of the particle delivery device include, but are not limited to hunger relief (e.g., in the emergency conditions of a famine) and for animal feedings.

Terms and phrases including “inhalable”, “exhalable”, “inhalation”, “exhalation”, “breathable”, “respiration”, “respirable”, “aspiration”, “inspiration”, “expiration”, “sip”, “sipping”, “sucking”, and others, have been used throughout this disclosure—or could have been used, as exact or approximate equivalents—to describe certain aspects of the disclosed embodiments. It should be noted that the definitions of each of these terms and phrases must be understood based on context and other relevant information herein. The precise definitions as understood in certain fields (e.g., medicine, anatomy, mechanical engineering, etc.) may not always be applicable in part or in whole.

Throughout the disclosure, “mouthpiece” and “first member” have been used interchangeably to describe function and/or structure of the components of the aerosolizing device described herein, and should be understood as being interchangeable for the descriptions as provided.

In addition, throughout this disclosure, “aerosol”, and similar terms (including singular and plural usages), are intended to refer to “a gaseous suspension of fine solid or liquid particles” (“aerosol” as defined in the American Heritage Dictionary online, 2011). For example, a dry powder that can be suspended in an air flow and transported via the airflow, as with the devices and payloads described herein, is considered to be within this definition. As another example, a plurality of liquid droplets substantially suspended in air as the result of ultrasonic agitation of a liquid, is also considered to be within this definition. Other examples of aerosols, and other relevant uses of such terms, would be evident to those skilled in the art; these examples and definition are therefore meant as clarification and in no way are intended to limit the scope or applicability of the terms as used herein.

### EXAMPLES

The examples below are provided herein for illustrative purposes and are not intended to be restrictive.

### Example 1

### Oral Hygiene/Breath Freshener, Serving Size 200 Milligrams

| Ingredient List | % w/w |
|---|---|
| Thymol Range of ingredient | 0.064% up to 64% |
| Menthol Range of ingredient | 0.04% up to 4% |
| Eucalyptus powder | 0.092% up to 9% |
| Vitamin B | 3% |
| Vitamin C | 5-30% |
| Maltodextrin Range of ingredient | 12-20% |
| Xylitol Range of ingredient | 15-35% |
| Sodium Bicarbonate (whitener) range | 10-20% |

### Example 2

### Oral Hygiene/Breath Freshener, Serving Size 200 Milligrams

| Ingredient List | % w/w |
|---|---|
| Thymol Range of ingredient | 0.064% up to 6.4% |
| Menthol Range of ingredient | 0.04% up to 4% |
| Eucalyptus powder | 0.092% up to 9% |
| Vitamin B | 3% |
| Vitamin C | 5-30% |
| Maltodextrin Range of ingredient | 12-20% |
| Xylitol Range of ingredient | 15-35% |
| Sodium Bicarbonate (whitener) range | 10-20% |
| Peppermint | 5% |
| Sucralose | 2% |

### Example 3

### Oral Hygiene/Breath Freshener, Serving Size 200 Milligrams

| Ingredient List | % w/w |
|---|---|
| Thymol Range of ingredient | 0.064% up to 6.4% |
| Menthol Range of ingredient | 0.04% up to 4% |
| Eucalyptus powder | 0.092% up to 9% |
| Vitamin B | 3% |
| Vitamin C | 5-30% |
| Maltodextrin Range of ingredient | 12-20% |
| Xylitol Range of ingredient | 15-35% |
| Sodium Bicarbonate (whitener) range | 10-20% |
| Peppermint | 5% |
| Sucralose | 2% |
| Zinc Chloride | |

### Example 4

### Oral Hygiene/Breath Freshener, Serving Size 200 Milligrams

| Ingredient List | % w/w |
|---|---|
| Thymol Range of ingredient | 0.064% up to 6.4% |
| Menthol Range of ingredient | 0.04% up to 4% |
| Eucalyptus powder | 0092% up to 9% |
| Vitamin B | 3% |
| Vitamin C | 5-30% |
| Maltodextrin Range of ingredient | 12-20% |
| Xylitol Range of ingredient | 15-35% |
| Sodium Bicarbonate (whitener) range | 10-20% |
| Peppermint | 5% |
| Sucralose | 2% |
| Methyl Salicylate | <0.5% |

### Example 5

### Oral Hygiene/Breath Freshener, Serving Size 200 Milligrams

| Ingredient List | % w/w |
|---|---|
| Thymol Range of ingredient | 0.064% up to 6.4% |
| Menthol Range of ingredient | 0.04% up to 4% |
| Eucolyptus powder | 0.092% up to 9% |
| Vitamin B | 3% |
| Vitamin C | 5-30% |
| Maltodextrin Range of ingredient | 12-20% |
| Xylitol Range of ingredient | 15-35% |
| Sodium Bicarbonate (whitener) range | 10-20% |
| Peppermint | 5% |
| Sucralose | 2% |
| Zinc Chloride | <1% |
| Methyl Salicylate | <0.5% |

### Example 6

### Novelty Powder: Stout Flavored Beer, Serving Size 300 Milligrams

| Ingredient List | % w/w |
|---|---|
| Caffeine | 35% |
| Coffee or cappacino | 8-10% |
| Chocolate | 20% |
| Sucralose | 1-2% |
| MonkFruit | 0-9% |
| Maltodextrin | 0-11% |
| Molasses | 5%-15% |
| Hops | 2-7% |
| Yeast (brewers) | 5% to 12% |
| Malt (german wheat) | 0-9% |

### Example 7

### Novelty Powder: Hefewiezen Flavored Beer, Serving Size 300 Milligrams

| Ingredient List | % w/w |
|---|---|
| Caffeine | 35% |
| Sucralose | 1-2% |
| MonkFruit | 0-9% |
| Maltodextrin | 0-11% |
| Molasses | 5%-15% |
| Hops | 2-7% |
| Yeast (brewers) | 5% to 12% |
| Malt (german wheat) | 0-20% |
| Coriander | 2% |
| Lemon | 10% |

### Example 8

### Sleep Aid: Melatonin Formula (DS) Serving Size 300 mg

| Ingredient List | % w/w |
|---|---|
| Melatonin | 1% |
| Chocolate | 50% |
| Sugar | 50% |

### Example 9

### Pharmaceutical Compound: Ciprofloxacin, Serving Size 500

| Ingredient List | % w/w |
|---|---|
| Ciprofloxacin | 50% |
| Cherry flavor | 24% |
| Monkfruit extract | 9% |
| Sucralose | 2% |
| Glycyrrihizine | 5% |
| Maltodextrin | 10% |

### Example 10

### OTC Allergy Pharmaceutical: Claritin, Serving Size 200 Milligrams

| Ingredient List | % w/w |
|---|---|
| Claritin | 2-5% |
| Grape flavoring | 30-40% |
| Sucralose | 1-2% |
| Tartaric acid | 10-15% |
| Sodium bicarbonate | 5-10% |
| Sorbitol | 10-15% |
| Maltodextrin | 30-40% |

### Example 11

### Diet Aid: Hoodia, Serving Size 400 Milligrams

| Ingredient List | % w/w |
|---|---|
| Hoodia | 50% |
| Grapefruit flavor | 25% |
| Citric acid | 10% |
| Thaumatin | 2% |
| Stevia | 1% |
| Maltodextrin | 12% |

### Example 12

### Vitamin/Mineral/Supplement Formula (a), 400 mg

| Ingredient List | % w/w |
|---|---|
| Sucralose | up to 6% |
| Lemon-lime flavor | up to 25% |
| Vitamin C (ascorbic acid) | 25% |
| Vitamin E (12 mg, as dl alpha tocopherol, 63.8%) | 3% |
| Vitamin B2 (riboflavin) | 0.4% |
| Vitamin A (beta carotene) 5000 IU(20%) | 1% |
| Lysine | up to 9% |
| Glutamine | 4% |
| Manganese gluconate | 0.75% |
| Magnesium citrate | up to 5% |
| Sodium bicarbonate | 5% |
| Potassium gluconate | 10% |
| Ginger | 1% |
| Camu powder | 5% |
| Zinc chloride (48%) | 2% |

### Example 13

### Vitamin/Mineral/Supplement Formula (b), 400 mg

| Ingredient List | % w/w |
|---|---|
| Sucralose | up to 6% |
| Green tea templar | up to 20% |
| Vitamin C (ascorbic acid) | 30% |
| Vitamin E (12 mg, as dl alpha tocopherol, 63.8%) | 3% |
| Vitamin B2 (riboflavin) | 0.4% |
| Vitamin A (beta carotene) 5000 IU(20%) | 1% |
| Lysine | up to 9% |
| Glutamine | 4% |
| Manganese gluconate | 0.75% |
| Magnesium citrate | up to 5% |
| Sodium bicarbonate | 5% |
| Potassium gluconate | 10% |
| Ginger | 1% |
| Camu powder | 5% |
| Zinc chloride (48%) | 2% |

### Example 14

### Vitamin/Mineral/Supplement Formula (c), 400 mg

| Ingredient List | % w/w |
|---|---|
| Grape flavoring | 4% |
| Green tea templar | 25% |
| Vitamin C (ascorbic acid) | 35% |
| Vitamin E (12 mg, as dl alpha tocopherol, 63.8%) | 3% |
| Vitamin B2 (riboflavin) | 0.4% |
| Vitamin A (beta carotene) 5000 IU(20%) | 1% |
| Lysine | 8% |
| Glutamine | 4% |
| Manganese gluconate | 0.75% |
| Magnesium citrate | 5% |
| Sodium bicarbonate | 6% |
| Potassium gluconate | 9% |
| Ginger | 1% |
| Camu powder | 5.85% |
| Zinc chloride (48%) | 2% |

### Example 15

### Vitamin/Mineral/Supplement Formula (d), 400 mg

| Ingredient List | % w/w |
|---|---|
| Pomagranate flavoring | 4% |
| Green tea templar | 25% |
| Vitamin C (ascorbic acid) | 35% |
| Vitamin E (12 mg, as dl alpha tocopherol, 63.8%) | 3% |
| Vitamin B2 (riboflavin) | 0.4% |
| Vitamin A (beta carotene) 5000 IU(20%) | 1% |
| Lysine | 8% |
| Glutamine | 4% |
| Manganese gluconate | 0.75% |
| Magnesium citrate | 5% |
| Sodium | 6% |
| Potassium gluconate | 9% |
| Ginger | 1% |
| Camu powder | 5.85% |
| Zinc (48%) | 2% |

### Example 15

### Vitamin/Mineral/Supplement Formula (e), 400 mg

| Ingredient List | % w/w |
|---|---|
| Orange flavoring | 4% |
| Green tea templar | 25% |
| Vitamin C (ascorbic acid) | 35% |
| Vitamin E (12 mg, as dl alpha tocopherol, 63,8%) | 3% |
| Vitamin B2 (riboflavin) | 0.4% |
| Vitamin A (beta carotene) 5000 IU(20%) | 1% |
| Lysine | 8% |
| Glutamine | 4% |
| Manganese gluconate | 0.75% |
| Magnesium citrate | 5% |
| Sodium bicarbonate | 6% |
| Potassium gluconate | 9% |
| Ginger | 1% |
| Camu powder | 5.85% |
| Zinc chloride (48%) | 2% |

### Example 16

### Energy (Caffeine) Formula (Grape), 300 mg

| Ingredient List | % w/w |
|---|---|
| Premix (see herein) | 47.67% |
| Citric acid | 9% |
| Sodium bicarbonate | 6% |
| Grape flavor | 20% |
| Monk fruit extract | 10% |
| Sucralose | 3% |
| Maltodextrine | 4.33% |

### Example 17

### Energy (Caffeine) Formula (Strawberry 1), 300 mg

| Ingredient List | % w/w |
|---|---|
| Premix (see herein) | 47.67% |
| Citric acid | 7.67% |
| Sodium bicarbonate | 5.667% |
| Strawberry flavor (Givaudan) | 25% |
| Talin | 1.5% |
| Sucralose | 43% |
| Maltodextrine | 8.499% |

### Example 18

### Energy (Caffeine) Formula (Strawberry 2), 300 mg

| Ingredient List | % w/w |
|---|---|
| Premix (see herein) | 47.67% |
| Citric acid | 7.67% |
| Sodium bicarbonate | 5.667% |
| Strawberry flavor (natural) | 24% |
| Monk | 12% |
| Sucralose | 3% |

### Example 19

### Ener (Caffeine) Formula (Strawberry 2), 300 mg

| Ingredient List | % w/w |
|---|---|
| Premix (see herein) | 47.67% |
| Citric acid | 7.67% |
| Sodium bicarbonate | 5.667% |
| Strawberry flavor (Givaudan) | 25% |
| Talin | 1.5% |
| Sucralose | 43% |
| Maltodextrine | 8.499% |

### Example 20

### Energy (Caffeine) Formula (Cherry), 300 mg

| Ingredient List | % w/w |
|---|---|
| Premix (see herein) | 47.67% |
| Citric acid | 5.67% |
| Sodium bicarbonate | 7.667% |
| Cherry flavor (Givaudan) | 20% |
| Monk fruit extract | 15% |
| Sucralose | 4% |

### Example 21

### Energy (Caffeine) Formula (Coffee), 300 mg

| Ingredient List | % w/w |
|---|---|
| Premix (see herein) | 47.67% |
| Barry cocoa | 7% |
| Monk fruit extract | 10% |
| Stevia | 2% |
| Isomalt | 8.33% |
| Expresso | 10% |
| Coffee | 15% |

### Example 22

### Energy (Caffeine) Formula (Watermelon 1), 300 mg

| Ingredient List | % w/w |
|---|---|
| Premix (see herein) | 47.67% |
| Malic acid | 5.66% |
| Sodium bicarbonate | 7.667% |
| Watermelon flavor (natural) | 20% |
| Monk fruit extract | 15% |
| Sucralose | 4% |

### Example 23

### Energy (Caffeine) Formula (Watermelon 2), 300 mg

| Ingredient List | % w/w |
|---|---|
| Premix (see herein) | 47.67% |
| Malic acid | 5.66% |
| Sodium bicarbonate | 7.667% |
| Watermelon flavor (natural + artificial) | 20% |
| Monk fruit extract | 15% |
| Sucralose | 4% |

An exemplary caffeine-based formulation, intended for delivery by an aerosol delivery apparatus, was created in accordance with the following recipe. In the following embodiments, a “pre-mix” formulation containing caffeine, B vitamins, and maltodextrin, was prepared separately. In formulations using the same pre-mix, it was desirable—per 300 mg dose of each powder—to have about 100 mg caffeine, the FDA daily values of each of the B vitamins, and a quantity of maltodextrin suitable for taste enhancement. As an option, additional maltodextrin was further added to improve taste. Similar premix formulations can be used in other embodiments described and referenced herein.

### Example 24

### Energy (Caffeine) Formula (Lime), Approximately 300 mg Total

| | |
|---|---|
| Premix: | |
| Caffeine | 108.0 mg |
| Vitamin B3 (niacin | 23.0 mg |
| Vitamin B6 (pyroxidine) | 2.9 mg |
| Vitamin B12 (cyanocobalamin) | 0.009 mg |
| Maltodextrin | 9.4 mg |
| Thaumatin | 4.5 mg |
| Stevia Rebaudioside A 97% | 3.0 mg |
| Lime flavoring agent | 30.0 mg |
| Additional maltodextrin | 41.5 mg |
| Wixon Masking Powder | 3.0 mg |
| Citric Acid | 45.0 mg |
| Sodium bicarbonate | 30.0 mg |

### Example 25

### Large Batch Energy Formulation (Caffeine with Lime Flavoring)

| | |
|---|---|
| Premix (see herein) | 28.6 kgs |
| Monk fruit extract | 5.4 kgs |
| Sucralose | 1.2 kgs |
| Lime flavoring | 7.2 kgs |
| Maltodextrin | 2.6 kgs |
| Sodium bicarbonate | 6.0 kgs |
| Citric acid | 9.0 kgs |

What is claimed is:

1. A carriage device for retaining a cartridge, the device comprising:
a first end defining an outlet port and configured to be detachably connected to a mouthpiece;
a second end defining an inlet port that is configured to align with an inlet port of the cartridge, the second end being opposed to the first end;
a sidewall connecting the first end and the second end, the sidewall defining an access port that is configured to permit passage of the cartridge therethrough;
a reservoir defined between the first end, second end and the sidewall, the reservoir configured to communicate with the inlet port, the outlet port and the access port and to receive the cartridge; and
an access port closure connected to the sidewall and movable between an open configuration and a closed configuration;
wherein when a cartridge is disposed in the reservoir, an air flow bypass passage is formed between the cartridge and an interior surface of the device.

2. The device of claim 1, wherein the access port closure is configured to rotate about a rotational axis that is parallel to a longitudinal axis of the device when moving between the open configuration and the closed configuration.

3. The device of claim 1, wherein the second end further comprises an opening that is configured to facilitate cartridge removal from the reservoir.

4. The device of claim 1, further comprising the mouthpiece connected to the first end, and the cartridge disposed in the reservoir, wherein the mouthpiece, the device and the cartridge cooperate to provide a flow rate through an interior of the device of between about ten (10) liters per minute and about sixty (60) liters per minute at a vacuum pressure of about four kiloPascals.

5. The device of claim 1, wherein the device is configured to be detachably connected to the mouthpiece by a press fit, a magnetic retaining mechanism, a twist mechanism, a snap mechanism, a screw mechanism, a bayonet mount mechanism, or combinations thereof.

6. The device of claim 1, further comprising the cartridge, the cartridge being at least one of edible and biodegradable and comprising a housing defining a cartridge reservoir, and an edible, aerosolizable powder disposed in the reservoir.

7. The device of claim 6, wherein the cartridge reservoir has a volume capacity of between about ten (10) milligrams to about two (2) grams of a product.

8. The device of claim 6, wherein the cartridge is formed from at least one of the group consisting of a starch, a grain-based food, a vegetable, a meat, a fruit, a dairy product, a sugary food, a nut, a confection, a plant product, processed edible products thereof, and synthetic edible products thereof.

9. The device of claim 6, wherein the cartridge is formed from at least one of the group consisting of chocolate, bread, fruit, sugar, meat, pasta, and processed forms thereof.

10. The device of claim 6, wherein the cartridge and the aerosolizable powder contained in the cartridge are comprised of at least one of the group selected from a vitamin, a mineral, and a supplement.

11. The device of claim 10, wherein the amount of vitamin, mineral or supplement provided by the cartridge, and the amount of vitamin, mineral, or supplement comprising the aerosolizable powder, in sum provide a quantity corresponding to an adult minimum daily requirement for the vitamin, mineral or supplement.

12. The device of claim 6, wherein the cartridge is biodegradable and is formed from at least one of the group consisting of a polyester, a polyhydroxyalkanoate, a polyanhydride, a polycaprolactone, a polydiaxonone, a polyglycolide, a polyhydroxybutyrate, a polylactic acid, a polypropylene carbonate, a polylactic-co-glycolic acid, a poly(3-hydroxybutyrate-co-3-hydroxyvalerate), a polyvinyl alcohol, a starch derivative, a cellulose derivative, a cellulose ester, a cellophane, an enhanced biodegradable plastic, and compositional variants thereof.

13. The device of claim 6, wherein a channel is provided on an exterior surface of the cartridge housing, and the channel is substantially parallel to a longitudinal axis of the device and configured to be in fluid communication with the device outlet port and device inlet port when the cartridge is disposed in the device reservoir, the channel operable as an air bypass passage when the access port closure is substantially in the closed configuration.

14. A carriage device for retaining a cartridge, the device comprising:
a first end configured to be detachably connected to a mouthpiece;
an open second end opposed to the first end;
an axis extending between the first end and the second end;
a sidewall extending between the first end and the second end;
a reservoir defined between the first end, the second end and the sidewall, the reservoir configured to receive the cartridge;
a first plunger, connected to the second end, the first plunger configured to be moveable relative to the sidewall along the axis; and
a second plunger, connected to the first plunger, the second plunger configured to move relative to the first plunger along the axis.

15. The carriage device of claim 14, wherein, the first plunger is configured to secure the cartridge within the reservoir.

16. The carriage device of claim 14, wherein the second plunger is configured to puncture the cartridge.

17. The device of claim 14, further comprising an awl located within the reservoir, the awl extending in parallel to the axis, wherein the awl is operable to puncture the cartridge.

18. The device of claim 14, wherein the sidewall includes an access port.

19. The device of claim 14, wherein the first plunger is releasably connected to the second end by a press fit, a magnetic retaining mechanism, a twist mechanism, a snap mechanism, a screw mechanism, a bayonet mount mechanism, or combinations thereof.

20. The device of claim 14, wherein the first plunger comprises a hollow cylinder defining a first cylinder end releasably connected to the second end and including an inwardly protruding lip circumscribing the inner surface substantially parallel to the first cylinder end.

21. The device of claim 14, wherein the second plunger comprises a second plunger first end disposed perpendicularly to the axis, and arms protruding from the second plunger first end, and wherein the arms are configured to engage the lip of the first plunger and permit movement of the second plunger along the axis.

22. The device of claim 14, wherein an outer surface of the first end is configured to define a bypass port between the first end and a surface of a mouthpiece when the carriage is assembled with the mouthpiece.

23. The device of claim 14, further comprising the mouthpiece connected to the device first end, and the cartridge disposed in the reservoir, wherein the mouthpiece, the device and the cartridge cooperate to provide a flow rate through the device of between about ten (10) liters per minute and about sixty (60) liters per minute at a vacuum pressure of about four kiloPascals.

24. The device of claim 14, wherein the mouth piece and the device are configured to be releasably connected by a press fit, a magnetic retaining mechanism, a twist mechanism, a snap mechanism, a screw mechanism, a bayonet mount mechanism, or combinations thereof.

25. The device of claim 14, further comprising the cartridge, wherein the cartridge is at least one of edible and biodegradable and comprises a housing defining a cartridge reservoir, and an edible, aerosolizable powder disposed in the cartridge reservoir.

26. The device of claim 25, wherein the cartridge reservoir has a volume capacity of between about ten (10) milligrams to about two (2) grams of a product.

27. The device of claim 25, wherein the cartridge is formed from at least one of the group consisting of a starch, a grain-based food, a vegetable, a meat, a fruit, a dairy product, a sugary food, a nut, a confection, a plant product, processed edible products thereof, and synthetic edible products thereof.

28. The device of claim 25, wherein the cartridge is formed from at least one of the group consisting of chocolate, bread, fruit, sugar, meat, pasta, and processed forms thereof.

29. The device of claim 25, wherein the cartridge and the aerosolizable powder contained in the cartridge are comprised of at least one of the group consisting of a vitamin, mineral and supplement.

30. The device of claim 29, wherein the amount of vitamin, mineral or supplement provided by the cartridge, and the amount of vitamin, mineral, or supplement comprising the aerosolizable powder, provide a quantity corresponding to an adult minimum daily requirement for the vitamin, mineral, or supplement.

31. The device of claim 25, wherein the cartridge is biodegradable and is formed from at least one of the group consisting of a polyester, a polyhydroxyalkanoate, a polyanhydride, a polycaprolactone, a polydiaxonone, a polyglycolide, a polyhydroxybutyrate, a polylactic acid, a polypropylene carbonate, a polylactic-co-glycolic acid, a poly(3-hydroxybutyrate-co-3-hydroxyvalerate), a polyvinyl alcohol, a starch derivative, a cellulose derivative, a cellulose ester, a cellophane, an enhanced biodegradable plastic, and compositional variants thereof.

32. A carriage device for retaining a cartridge, the device comprising:
a first end configured to be releasably connected to a mouthpiece;
a second end opposed to the first end,
a sidewall extending between the first end and the second end;
a partition that is disposed between the first end and the second end and extends between opposed inner surfaces of the sidewall to segregate an interior space of the carriage into a first portion and a second portion; and
a piercing tool disposed in the second portion.

33. The device of claim 32 wherein the first end includes legs equidistantly spaced about a circumference of the sidewall, the legs configured for engagement with a surface of the mouthpiece.

34. The device of claim 33, wherein the first portion is defined between the legs and the partition, and forms a reservoir configured to receive a cartridge.

35. The device of claim 32, wherein the partition includes openings that provide fluid communication between the first portion and the second portion.

36. The device of claim 32, further comprising the mouthpiece releasably connected to the first end, and the cartridge disposed in the first portion, wherein the mouthpiece, the device and the cartridge cooperate to provide a flow rate through the device of between about ten (10) liters per minute and about sixty (60) liters per minute at a vacuum pressure of about four kiloPascals.

37. The device of claim 36, wherein the mouth piece and the device are configured to be releasably connected by a press lit, a magnetic retaining mechanism, a twist mechanism, a snap mechanism, a screw mechanism, a bayonet mount mechanism, or combinations thereof.

38. The device of claim 32, further comprising the cartridge, the cartridge being at least one of edible and biodegradable, the cartridge comprising a housing defining a cartridge reservoir, and an edible, aerosolizable powder disposed in the cartridge reservoir.

39. The device of claim 38, wherein the cartridge reservoir has a volume capacity of between about ten (10) milligrams to about two (2) grams of a product.

40. The device of claim 38, wherein the cartridge is formed from at least one of the group consisting of a starch, a grain-based food, a vegetable, a meat, a fruit, a dairy product, a sugary food, a nut, a confection, a plant product, processed edible products thereof, and synthetic edible products thereof.

41. The device of claim 38, wherein the cartridge is formed from at least one of the group consisting of chocolate, bread, fruit, sugar, meat, pasta, and processed forms thereof.

42. The device of claim 38, wherein the cartridge and the aerosolizable powder contained in the cartridge are comprised of at least one of the group consisting of the group consisting of a vitamin, a mineral, and a supplement.

43. The device of claim 42, wherein the amount of vitamin, mineral, or supplement provided by the cartridge, and the amount of vitamin, mineral, or supplement comprising the aerosolizable powder provide a quantity corresponding to an adult minimum daily requirement for the vitamin, mineral or supplement.

44. The device of claim 38, wherein the cartridge is biodegradable and is formed from at least one of the group consisting of a polyester, a polyhydroxyalkanoate, a polyanhydride, a polycaprolactone, a polydiaxonone, a polyglycolide, a polyhydroxybutyrate, a polylactic acid, a polypropylene carbonate, a polylactic-co-glycolic acid, a poly(3-hydroxybutyrate-co-3-hydroxyvalerate), a polyvinyl alcohol, a starch derivative, a cellulose derivative, a cellulose ester, a cellophane, an enhanced biodegradable plastic, and compositional variants thereof.
